SEQUENCE LISTING (2 sequences)

SEQ ID NO: 1 - RNA, 3552 nt, Homo sapiens
  source 1..3552: mol_type = mRNA
aacgctccgccggcgccaatttcaaacagcggaacaaactgaaagctccggtgccagaccccacccccggccccggcccgggaccccctcccctcccgggatcccccggggttcccaccccgcccgcaccgccggggacccggccggtccggcgcgagcccccgtccggggccctggctcggcccccaggttggaggagcccggagcccgccttcggagctacggcctaacggcggcggcgactgcagtctggagggtccacacttgtgattctcaatggagagtgaaaacgcagattcataatgaaaactagcccccgtcggccactgattctcaaaagacggaggctgccccttcctgttcaaaatgccccaagtgaaacatcagaggaggaacctaagagatcccctgcccaacaggagtctaatcaagcagaggcctccaaggaagtggcagagtccaactcttgcaagtttccagctgggatcaagattattaaccaccccaccatgcccaacacgcaagtagtggccatccccaacaatgctaatattcacagcatcatcacagcactgactgccaagggaaaagagagtggcagtagtgggcccaacaaattcatcctcatcagctgtgggggagccccaactcagcctccaggactccggcctcaaacccaaaccagctatgatgccaaaaggacagaagtgaccctggagaccttgggaccaaaacctgcagctagggatgtgaatcttcctagaccacctggagccctttgcgagcagaaacgggagacctgtgcagatggtgaggcagcaggctgcactatcaacaatagcctatccaacatccagtggcttcgaaagatgagttctgatggactgggctcccgcagcatcaagcaagagatggaggaaaaggagaattgtcacctggagcagcgacaggttaaggttgaggagccttcgagaccatcagcgtcctggcagaactctgtgtctgagcggccaccctactcttacatggccatgatacaattcgccatcaacagcactgagaggaagcgcatgactttgaaagacatctatacgtggattgaggaccactttccctactttaagcacattgccaagccaggctggaagaactccatccgccacaacctttccctgcacgacatgtttgtccgggagacgtctgccaatggcaaggtctccttctggaccattcaccccagtgccaaccgctacttgacattggaccaggtgtttaagccactggacccagggtctccacaattgcccgagcacttggaatcacagcagaaacgaccgaatccagagctccgccggaacatgaccatcaaaaccgaactccccctgggcgcacggcggaagatgaagccactgctaccacgggtcagctcatacctggtacctatccagttcccggtgaaccagtcactggtgttgcagccctcggtgaaggtgccattgcccctggcggcttccctcatgagctcagagcttgcccgccatagcaagcgagtccgcattgcccccaaggtgctgctagctgaggaggggatagctcctctttcttctgcaggaccagggaaagaggagaaactcctgtttggagaagggttttctcctttgcttccagttcagactatcaaggaggaagaaatccagcctggggaggaaatgccacacttagcgagacccatcaaagtggagagccctcccttggaagagtggccctccccggccccatctttcaaagaggaatcatctcactcctgggaggattcgtcccaatctcccaccccaagacccaagaagtcctacagtgggcttaggtccccaacccggtgtgtctcggaaatgcttgtgattcaacacagggagaggagggagaggagccggtctcggagaaaacagcatctactgcctccctgtgtggatgagccggagctgctcttctcagaggggcccagtacttcccgctgggccgcagagctcccgttcccagcagactcctctgaccctgcctcccagctcagctactcccaggaagtgggaggaccttttaagacacccattaaggaaacgctgcccatctcctccaccccgagcaaatctgtcctccccagaacccctgaatcctggaggctcacgcccccagccaaagtagggggactggatttcagcccagtacaaacctcccagggtgcctctgaccccttgcctgaccccctggggctgatggatctcagcaccactcccttgcaaagtgctcccccccttgaatcaccgcaaaggctcctcagttcagaacccttagacctcatctccgtcccctttggcaactcttctccctcagatatagacgtccccaagccaggctccccggagccacaggtttctggccttgcagccaatcgttctctgacagaaggcctggtcctggacacaatgaatgacagcctcagcaagatcctgctggacatcagctttcctggcctggacgaggacccactgggccctgacaacatcaactggtcccagtttattcctgagctacagtagagccctgcccttgcccctgtgctcaagctgtccaccatcccgggcactccaaggctcagtgcaccccaagcctctgagtgaggacagcaggcagggactgttctgctcctcatagctccctgctgcctgattatgcaaaagtagcagtcacaccctagccactgctgggaccttgtgttccccaagagtatctgattcctctgctgtccctgccaggagctgaagggtgggaacaacaaaggcaatggtgaaaagagattaggaaccccccagcctgtttccattctctgcccagcagtctcttaccttccctgatctttgcagggtggtccgtgtaaatagtataaattctccaaattatcctctaattataaatgtaagcttatttccttagatcattatccagagactgccagaaggtgggtaggatgacctggggtttcaattgacttctgttccttgcttttagttttgatagaagggaagacctgcagtgcacggtttcttccaggctgaggtacctggatcttgggttcttcactgcagggacccagacaagtggatctgcttgccagagtcctttttgcccctccctgccacctccccgtgtttccaagtcagctttcctgcaagaagaaatcctggttaaaaaagtcttttgtattgggtcaggagttgaatttggggtgggaggatggatgcaactgaagcagagtgtgggtgcccagatgtgcgctattagatgtttctctgataatgtccccaatcataccagggagactggcattgacgagaactcaggtggaggcttgagaaggccgaaagggcccctgacctgcctggcttccttagcttgcccctcagctttgcaaagagccaccctaggccccagctgaccgcatgggtgtgagccagcttgagaacactaactactcaataaaagcgaaggtggacatgc

SEQ ID NO: 2 - protein, 763 aa, Homo sapiens
MKTSPRRPLILKRRRLPLPVQNAPSETSEEEPKRSPAQQESNQAEASKEVAESNSCKFPAGIKIINHPTMPNTQVVAIPNNANIHSIITALTAKGKESGSSGPNKFILISCGGAPTQPPGLRPQTQTSYDAKRTEVTLETLGPKPAARDVNLPRPPGALCEQKRETCADGEAAGCTINNSLSNIQWLRKMSSDGLGSRSIKQEMEEKENCHLEQRQVKVEEPSRPSASWQNSVSERPPYSYMAMIQFAINSTERKRMTLKDIYTWIEDHFPYFKHIAKPGWKNSIRHNLSLHDMFVRETSANGKVSFWTIHPSANRYLTLDQVFKPLDPGSPQLPEHLESQQKRPNPELRRNMTIKTELPLGARRKMKPLLPRVSSYLVPIQFPVNQSLVLQPSVKVPLPLAASLMSSELARHSKRVRIAPKVLLAEEGIAPLSSAGPGKEEKLLFGEGFSPLLPVQTIKEEEIQPGEEMPHLARPIKVESPPLEEWPSPAPSFKEESSHSWEDSSQSPTPRPKKSYSGLRSPTRCVSEMLVIQHRERRERSRSRRKQHLLPPCVDEPELLFSEGPSTSRWAAELPFPADSSDPASQLSYSQEVGGPFKTPIKETLPISSTPSKSVLPRTPESWRLTPPAKVGGLDFSPVQTSQGASDPLPDPLGLMDLSTTPLQSAPPLESPQRLLSSEPLDLISVPFGNSSPSDIDVPKPGSPEPQVSGLAANRSLTEGLVLDTMNDSLSKILLDISFPGLDEDPLGPDNINWSQFIPELQ

(19) United States
(12) Patent Application Publication
(10) Pub. No.: US 2026/0217691 A1
(43) Pub. Date: Jul. 30, 2026

(54) FOXM1 INHIBITORS AND THEIR USE IN TREATING CANCERS

(71) Applicants: THE BOARD OF TRUSTEES OF THE UNIVERSITY OF ILLINOIS, URBANA, IL (US); UNIVERSITY OF PITTSBURGH - OF THE COMMONWEALTH SYSTEM OF HIGHER EDUCATION, Pittsburgh, PA (US); UNIVERSITY OF GRONINGEN, Groningen (NL)

(72) Inventors: CARLOS JAIME CAMACHO, PITTSBURGH, PA (US); ALEXANDER DÖMLING, SOLINGEN (DE); ANDREI GARTEL, CHICAGO, IL (US)

(21) Appl. No.: 19/142,285

(22) PCT Filed: Dec. 21, 2023
(86) PCT No.: PCT/US2023/085415

§ 371 Date: Jun. 23, 2025

Related U.S. Application Data
(60) Provisional application No. 63/477,021, filed on Dec. 23, 2022.
(60) Provisional application No. 63/486,734, filed on Feb. 24, 2023.

(51) Int. Cl. C07D 405/14; A61K 31/138; A61K 31/337; A61K 31/513; A61K 31/5377; A61K 31/635; A61K 31/704; A61K 31/7068; A61K 33/243; A61P 35/00; A61P 35/02

(52) U.S. Cl. CPC C07D 405/14; A61K 31/138; A61K 31/337; A61K 31/513; A61K 31/5377; A61K 31/635; A61K 31/704; A61K 31/7068; A61K 33/243; A61P 35/00; A61P 35/02

(57) ABSTRACT

Provided herein is therapy for use in treating cancers, for example as an adjuvant therapy for use in conjunction with a chemotherapeutic agent in treatment of cancer, such as a cancer in which FOXM1 is overexpressed, including AML and solid tumors. FOXM1 inhibitory compounds also are provided herein including pharmaceutical compositions comprising the compounds.

### CROSS REFERENCE TO RELATED APPLICATIONS

This application claims priority to U.S. Provisional Patent Application No. 63/477,021 filed Dec. 23, 2022, and U.S. Provisional Patent Application No. 63/486,734 filed Feb. 24, 2023, each of which is incorporated herein by reference in its entirety.

### STATEMENT REGARDING FEDERAL FUNDING

This invention was made with government support under Grant No. GM097082, awarded by the National Institutes of Health. The government has certain rights in the invention.

### REFERENCE TO A SEQUENCE LISTING

The Sequence Listing associated with this application is filed in electronic format via Patent Center and is hereby incorporated by reference into the specification in its entirety. The name of the XML file containing the Sequence Listing is 06527-2308517.xml. The size of the XML file is 6,914 bytes and the XML file was created on Dec. 21, 2023.

Forkhead box (FOX) protein M1 (FOXM1) is a transcription factor with pronounced pro-oncogenic functions (see, e.g., Gene ID: 2305; HGNC:3818; NCBI Reference Sequence: NM_021953.4 (FIGS. 1A and 1B; SEQ ID NOS. 1 and 2). It is overexpressed in the majority of human cancers and impacts all hallmark tumor aspects, including proliferation, survival, metastasis, inflammation, angiogenesis, and treatment resistance. Due to this, FOXM1 serves as a crucial regulator of tumor development, and its overexpression portends a poor prognosis for patients, promoting aggressive tumor phenotype and high resistance to current therapeutic approaches (see, e.g., Chesnokov, M. S. et al. Novel FOXM1 inhibitor identified via gene network analysis induces autophagic FOXM1 degradation to overcome chemoresistance of human cancer cells, Cell Death and Disease (2021) 12:704; doi.org/10.1038/s41419-021-03978-0). Human cancers displaying increased expression of FOXM1 include, for example: ovarian cancer, breast cancer, prostate cancer, hepatoma, angiosarcoma, colorectal cancer, melanoma, lung cancer, and gastric cancer (see, e.g., Bai et al. (Liao G B, et al. Regulation of the master regulator FOXM1 in cancer. Cell Commun Signal. 2018 Sep. 12; 16(1):57) and Kalathil D, et al. FOXM1 and Cancer: Faulty Cellular Signaling Derails Homeostasis. Front Oncol. 2021 Feb. 15; 10:626836).

Pharmacological inhibition of FOXM1 is a promising approach but has proven to be challenging. Identification of pharmacological inhibitors of FOXM1 is therefore desirable.

### SUMMARY

According to a first aspect or embodiment, a compound is provided, comprising the structure:

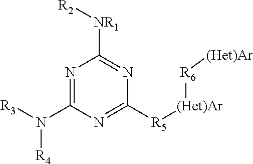

wherein,
(Het)Ar is (hetero)aryl;
R₁ is: H or alkyl, e.g., C₁₋₆ alkyl or C₁₋₃ alkyl;
R₂ is: optionally substituted (hetero)aryl;
R₃ and R₄ are independently: H, Me, Et, cyclopropyl, or R₃ and R₄ taken together are:
—CH₂CH₂—O—CH₂CH₂—, —CH₂CH₂—N(H)—CH₂CH₂—, —CH₂CH₂—S—CH₂CH₂—, —CH₂CH₂—N(Me)-CH₂CH₂—, —CH₂CH₂—N(Et)-CH₂CH₂—, —CH₂CH₂—S(O)—CH₂CH₂—, —CH₂CH₂—S(O)₂—CH₂CH₂—, —CH₂CH₂—N(H)—CH₂CH₂CH₂—, or —CH₂CH₂—N(Me)-CH₂CH₂CH₂—; and
R₅ and R₆ are, independently, saturated or unsaturated (hetero)cyclopentyl having the structure

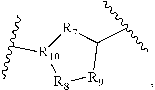

where R₇, R₈, R₉, and R₁₀ are, independently, C, O, N, or S, optionally comprising one or two double bonds in the (hetero)cyclopentyl ring;
or
an amide bond, e.g.,

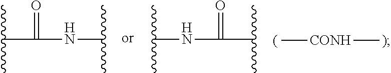

or
an ester bond, e.g.

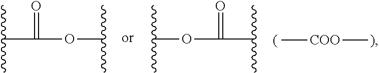

or a pharmaceutically-acceptable salt thereof, including stereoisomers thereof and mixtures of stereoisomers thereof. According to a further aspect or embodiment, a composition is provided, comprising the compound, and a pharmaceutically-acceptable excipient or carrier.

According to another aspect of embodiment of the invention, a method of treating a patient having a cancer is provided, comprising administering to the patient amounts of a chemotherapeutic agent and a compound as described in the preceding paragraph or STL427944 where the patient has a leukemia, effective to treat the cancer in the patient. In another aspect or embodiment, a method of increasing sensitivity of a cancer cell to a chemotherapeutic agent in a patient, e.g., having a leukemia, is provided, comprising administering to the patient an amount of the compound or STL427944 where the patient has a leukemia, effective to increase sensitivity of a cancer cell in the patient to the chemotherapeutic agent. Lastly, according to another aspect or embodiment, a method of treating a patient having a cancer is provided, comprising administering to the patient amounts of the compound, or STL427944 where the patient has a leukemia, effective to treat the disease in the patient.

The following numbered clauses outline various aspects or embodiments of the present invention.

Clause 1. A compound having the structure:

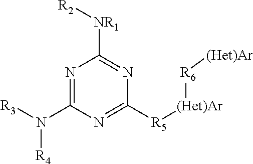

wherein,
(Het)Ar is (hetero)aryl;
R₁ is: H or alkyl, e.g., C₁₋₆ alkyl or C₁₋₃ alkyl;
R₂ is: optionally substituted (hetero)aryl;
R₃ and R₄ are independently: H, Me, Et, cyclopropyl, or R₃ and R₄ taken together are:
—CH₂CH₂—O—CH₂CH₂—, —CH₂CH₂—N(H)—CH₂CH₂—, —CH₂CH₂—S—CH₂CH₂—, —CH₂CH₂—N(Me)-CH₂CH₂—, —CH₂CH₂—N(Et)-CH₂CH₂—, —CH₂CH₂—S(O)—CH₂CH₂—, —CH₂CH₂—S(O)₂—CH₂CH₂—, —CH₂CH₂—N(H)—CH₂CH₂CH₂—, or —CH₂CH₂—N(Me)-CH₂CH₂CH₂—; and
R₅ and R₆ are, independently, saturated or unsaturated (hetero)cyclopentyl having the structure

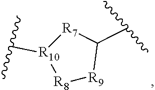

where R₇, R₈, R₉, and R₁₀ are, independently, C, O, N, or S, optionally comprising one or two double bonds in the (hetero)cyclopentyl ring;
or
an amide bond, e.g.,

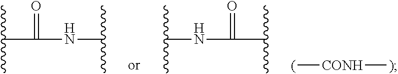

or
an ester bond, e.g.

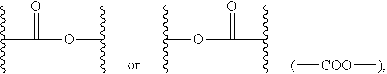

or a pharmaceutically-acceptable salt thereof, including stereoisomers thereof and mixtures of stereoisomers thereof.

Clause 2. The compound of claim 1, wherein R₂ is substituted phenyl, thiophenyl, oxazolyl, thiazolyl, isothiazolyl, furanoyl.

Clause 3. The compound of claim 1, wherein R₂ is substituted with one or more of: —H, -D, -Me, —CHD₂, —CDs, CH₂D, —F, —CF₃, —Cl, —CN, -Et, -iPr, or —OMe, or a combination of any of the preceding.

Clause 4. The compound of any one of claims 1-3, wherein R₁ is H.

Clause 5. The compound of claim 1, having the structure:

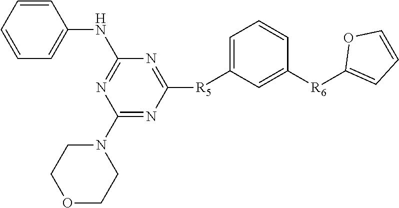

wherein,
R₅ is:
saturated or unsaturated cyclopentyl or heterocyclopentyl having the structure

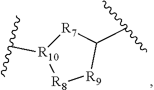

where R₇, R₈, R₉, and R₁₀ are, independently, C, O, N, or S; and
R₆ is:
saturated or unsaturated cyclopentyl or heterocyclopentyl having the structure

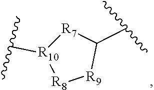

where R₇, R₈, R₉, and R₁₀ are, independently, C, O, N, or S;
an amide bond; or
an ester bond,
or a pharmaceutically-acceptable salt thereof, including stereoisomers thereof and mixtures of stereoisomers thereof.

Clause 6. The compound of claim 5, wherein R₅ is

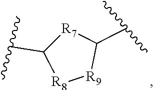

Clause 7. The compound of claim 6, wherein at least one of R₇, R₈, and R₉ are N, S, or O.

Clause 8. The compound of claim 5 or 6, wherein at least one of R₇, R₈, and R₉ are N.

Clause 9. The compound of any one of claims 1-5, wherein for R₅, at least one of R₇, R₈, R₉, and R₁₀ are N, S, or O.

Clause 10. The compound of any one of claims 1-5, wherein R₆ is

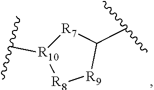

and at least one of R₇, R₈, R₉, and R₁₀ are N, S, or O.

Clause 11. The compound of any one of claims 1-5, wherein R₆ is

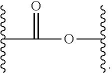 [SMILES: *OC(*)=O]

Clause 12. The compound of any one of claims 1-5, wherein R₆ is

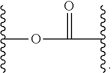 [SMILES: *OC(*)=O]

Clause 13. The compound of any one of claims 1-5, wherein R₆ is

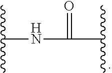 [SMILES: *NC(*)=O]

Clause 14. The compound of any one of claims 1-5, wherein R₆ is

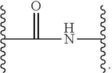 [SMILES: *NC(*)=O]

Clause 15. The compound of any one of claims 1-14, wherein R₅ is

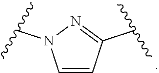 [SMILES: *c1ccn(*)n1]

Clause 16. The compound of any one of claim 1-14, wherein R₅ is a pyrazole ring.

Clause 17. The compound of any one of claims 1-14, wherein R₅ is a pyrrole, furan, thiophene, pyrazole, imidazole, oxazole, isooxazole, pyrrolidine, thiolane (tetrahydrothiophene), or tetrahydrofuran ring.

Clause 18. The compound of claim 1, having the structure:

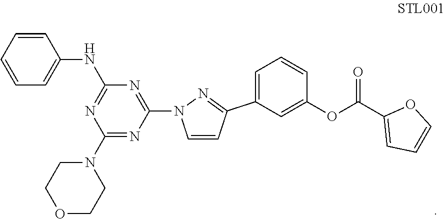 [SMILES: O=C(Oc1cccc(-c2ccn(-c3nc(Nc4ccccc4)nc(N4CCOCC4)n3)n2)c1)c1ccco1]

or a pharmaceutically-acceptable salt thereof, including stereoisomers thereof and mixtures of stereoisomers thereof.

Clause 19. A composition comprising the compound of any one of claims 1-18, and a pharmaceutically-acceptable excipient or carrier.

Clause 20. The composition of claim 19, in the form of a parenteral dosage form.

Clause 21. The composition of claim 19 or 20, further comprising a chemotherapeutic agent.

Clause 22. The composition of claim 21, wherein the chemotherapeutic agent is one or more of: abiraterone acetate, altretamine, amsacrine, anhydro vinblastine, auristatin, bafetinib, bexarotene, bicalutamide, BMS 184476, 2,3,4,5,6-pentafluoro-N-(3-fluoro-4-methoxyphenyl)benzene sulfonamide, bleomycin, bosutinib, busulfan, cachectin, cemadotin, chlorambucil, cyclophosphamide, 3′,4′-didehydro-4′-deoxy-8′-norvin-caleukoblastine, docetaxol, doxetaxel, carboplatin, carmustine (BCNU), chlorambucil, cisplatin, cryptophycin, cyclophosphamide, cytarabine, dacarbazine (DTIC), dactinomycin, daunorubicin, decitabine dolastatin, doxorubicin (adriamycin), etoposide, etoposide phosphate, 5-fluorouracil, finasteride, flutamide, gemcitabine, hydroxyurea, hydroxyureataxanes, ifosfamide, imatinib, irinotecan, liarozole, lonidamine, lomustine (CCNU), Enzalutamide, mechlorethamine (nitrogen mustard), melphalan, mitoxantrone, mivobulin isethionate, rhizoxin, sertenef, streptozocin, mitomycin, methotrexate, nilotinib, nilutamide, onapristone, oxaliplatin, paclitaxel, ponatinib, prednimustine, procarbazine, stramustine phosphate, tamoxifen, tasonermin, taxol, teniposide, topotecan, tretinoin, venetoclax, vinblastine, vincristine, vindesine sulfate, vinflunine, or a pharmaceutically acceptable salt or ester thereof.

Clause 23. A method of treating a patient having a cancer, comprising administering to the patient amounts of a chemotherapeutic agent and a compound of any one of claims 1-18 or STL427944 where the patient has a leukemia, effective to treat the cancer in the patient.

Clause 24. The method of claim 23, wherein the chemotherapeutic agent is one or more of: abiraterone acetate, altretamine, amsacrine, anhydro vinblastine, auristatin, bafetinib, bexarotene, bicalutamide, BMS 184476, 2,3,4,5,6-pentafluoro-N-(3-fluoro-4-methoxyphenyl)benzene sulfonamide, bleomycin, bosutinib, busulfan, cachectin, cemadotin, chlorambucil, cyclophosphamide, 3′,4′-didehydro-4′-deoxy-8′-norvin-caleukoblastine, docetaxol, doxetaxel, carboplatin, carmustine (BCNU), chlorambucil, cisplatin, cryptophycin, cyclophosphamide, cytarabine, dacarbazine (DTIC), dactinomycin, daunorubicin, decitabine dolastatin, doxorubicin (adriamycin), etoposide, etoposide phosphate, 5-fluorouracil, finasteride, flutamide, gemcitabine, hydroxyurea, hydroxyureataxanes, ifosfamide, imatinib, irinotecan, liarozole, lonidamine, lomustine (CCNU), Enzalutamide, mechlorethamine (nitrogen mustard), melphalan, mitoxantrone, mivobulin isethionate, rhizoxin, sertenef, streptozocin, mitomycin, methotrexate, nilotinib, nilutamide, onapristone, oxaliplatin, paclitaxel, ponatinib, prednimustine, procarbazine, stramustine phosphate, tamoxifen, tasonermin, taxol, teniposide, topotecan, tretinoin, venetoclax, vinblastine, vincristine, vindesine sulfate, vinflunine, or a pharmaceutically acceptable salt or ester thereof.

Clause 25. The method of claim 23 or 24, wherein the compound is STL001, or a pharmaceutically-acceptable salt thereof, including stereoisomers thereof and mixtures of stereoisomers thereof.

Clause 26. The method of any one of claims 23-25, wherein the cancer is a leukemia.

Clause 27. The method of claim 26, wherein the leukemia is AML.

Clause 28. The method of any one of claims 23-25, wherein the cancer is ovarian cancer, breast cancer, prostate cancer, hepatoma, angiosarcoma, colorectal cancer, melanoma, lung cancer, or gastric cancer and/or FOXM1 is overexpressed or abnormally expressed in cancer cells of the patient.

Clause 29. The method of any one of claims 23-25, wherein the cancer is:
ovarian cancer and the chemotherapeutic agent is doxorubicin;
colon cancer, and the chemotherapeutic agent is 5′-fluorouracil;
prostate cancer and the chemotherapeutic agent is paclitaxel;
tamoxifen-resistant breast cancer and the chemotherapeutic agent is tamoxifen; or
triple negative breast cancer and the chemotherapeutic agent is doxorubicin and/or cisplatin, and
wherein FOXM1 is overexpressed or abnormally expressed in cancer cells of the patient.

Clause 30. A method of increasing sensitivity of a cancer cell to a chemotherapeutic agent in a patient, e.g., having a leukemia, comprising administering to the patient an amount of a compound as claimed in any one of claims 1-18 or STL427944 where the patient has a leukemia, effective to increase sensitivity of a cancer cell in the patient to the chemotherapeutic agent.

Clause 31. The method of claim 30, wherein the cancer cell is a leukemia cell.

Clause 32. The method of claim 31, wherein the cancer cell is from a patient having AML (Acute Myeloid Leukemia), such as a cytogenetically normal-AML, such as with FLT3-WT and mutant NPM1.

Clause 33. The method of claim 30, wherein the cancer cell is a cancer cell of a patient having ovarian cancer, breast cancer, prostate cancer, hepatoma, angiosarcoma, colorectal cancer, melanoma, esophageal cancer, lung cancer, or gastric cancer and/or FOXM1 is overexpressed or abnormally expressed in cancer cells of the patient.

Clause 34. The method of claim 33, wherein the cancer is:
ovarian cancer and the chemotherapeutic agent is doxorubicin;
colon cancer, and the chemotherapeutic agent is 5′-fluorouracil;
prostate cancer and the chemotherapeutic agent is paclitaxel;
tamoxifen-resistant breast cancer and the chemotherapeutic agent is tamoxifen; or
triple negative breast cancer and the chemotherapeutic agent is doxorubicin and/or cisplatin, and
wherein FOXM1 is overexpressed or abnormally expressed in the cancer cells.

Clause 35. The method of claim 30, wherein the cancer cell is a solid tumor cancer cell.

Clause 36. The method of any one of claims 30-35, wherein the compound is STL001, or a pharmaceutically-acceptable salt thereof, including stereoisomers thereof and mixtures of stereoisomers thereof.

Clause 37. The method of any one of claims 30-33, wherein the chemotherapeutic agent is one or more of: abiraterone acetate, altretamine, amsacrine, anhydro vinblastine, auristatin, bafetinib, bexarotene, bicalutamide, BMS 184476, 2,3,4,5,6-pentafluoro-N-(3-fluoro-4-methoxyphenyl)benzene sulfonamide, bleomycin, bosutinib, busulfan, cachectin, cemadotin, chlorambucil, cyclophosphamide, 3′,4′-didehydro-4′-deoxy-8′-norvin-caleukoblastine, docetaxol, doxetaxel, carboplatin, carmustine (BCNU), chlorambucil, cisplatin, cryptophycin, cyclophosphamide, cytarabine, dacarbazine (DTIC), dactinomycin, daunorubicin, decitabine dolastatin, doxorubicin (adriamycin), etoposide, etoposide phosphate, 5-fluorouracil, finasteride, flutamide, gemcitabine, hydroxyurea, hydroxyureataxanes, ifosfamide, imatinib, irinotecan, liarozole, lonidamine, lomustine (CCNU), Enzalutamide, mechlorethamine (nitrogen mustard), melphalan, mitoxantrone, mivobulin isethionate, rhizoxin, sertenef, streptozocin, mitomycin, methotrexate, nilotinib, nilutamide, onapristone, oxaliplatin, paclitaxel, ponatinib, prednimustine, procarbazine, stramustine phosphate, tamoxifen, tasonermin, taxol, teniposide, topotecan, tretinoin, venetoclax, vinblastine, vincristine, vindesine sulfate, vinflunine, or pharmaceutically acceptable salts or esters thereof.

Clause 38. The method of any one of claims 23-37, wherein the patient is human.

Clause 39. A method of treating a patient having a cancer, comprising administering to the patient amounts of a compound of any one of claims 1-18, or STL427944 where the patient has a leukemia, effective to treat the disease in the patient.

Clause 40. The method of claim 39, wherein the compound is STL001, or a pharmaceutically-acceptable salt thereof, including stereoisomers thereof and mixtures of stereoisomers thereof.

Clause 41. The method of claim 39 or 40, wherein the disease is a cancer.

Clause 42. The method of claim 41, wherein the cancer is a leukemia.

Clause 43. The method of claim 42, wherein the leukemia is AML.

Clause 44. The method of claim 41, wherein the cancer is ovarian cancer, breast cancer, prostate cancer, hepatoma, angiosarcoma, colorectal cancer, melanoma, lung cancer, or gastric cancer and/or FOXM1 is overexpressed or abnormally expressed in cancer cells of the patient.

### BRIEF DESCRIPTION OF THE DRAWINGS

FIGS. 1A and 1B provide exemplary mRNA (FIG. 1A, NCBI Reference Sequence: NM_021953.4; SEQ ID NO. 1) and protein (FIG. 1B, NCBI Reference Sequence: NP_068772.2; SEQ ID NO. 2) sequences for human FOXM1 (transcript variant 2).

FIG. 2 depicts synthesis scheme for compound STL001 (AD-5), as described in Example 1.

FIG. 3 provides graphs of supercritical fluid chromatography for compound STL001 (AD-5), as prepared according to Example 1.

FIGS. 4A and 4B provide spectra for compound STL001 (AD-5), as prepared according to Example 1.

FIG. 5. FOXM1 is an independent predictor of chemotherapy resistance in intermediate risk CN-AML. (A) Bone marrow slides were stained with FOXM1 antibody and counterstained with hematoxylin. Images were analyzed utilizing the Aperio AT2 whole slide scanner and HALO 2.0 software. Two representative patient samples are shown (200× magnification) with high and low percentage of nuclei expressing FOXM1 with the corresponding markup images below that were quantified. (B) In an analysis of the patients who achieved a CR following chemotherapy there were 74 bone marrow samples with quantifiable FOXM1 expression. We found that patients needing more than 1 cycle of induction therapy had greater than a 2-fold increase in the percentage of nuclei expressing FOXM1 (mean 25.6% vs. 11.4% nuclei, p=0.004) in their diagnostic bone marrow. (C) Kaplan-Meier analysis for overall survival in 45 patients from a single institution in our cohort, stratified based on average nuclear intensity of FOXM1. FOXM1 hi patients have an inferior survival that approaches statistical significance (median 501 days vs. not reached, p=0.068).

FIG. 6. FOXM1 confers chemo-resistance in AML cell lines. (A) Cellular fractionation followed by immunoblot analysis shows nuclear localization of FOXM1 in KG-1 cells, originating from a patient with AML. (B) Targeting FOXM1 with shRNA increases sensitivity of KG-1 cells to cytarabine (AraC). An increase in cell-death is shown by caspase-3 cleavage after 24 hours exposure to the drug in KG-1 shFOXM1 cells. (C, D) Pharmacologic inhibition of FOXM1 using bortezomib or (E) thiostrepton shows down-regulation of FOXM1 and synergistic induction of apoptosis with chemotherapy drug cytarabine as detected by caspase-3 cleavage in KG-1 cells. (F) (left panel) The NPM1-mutated OCI-AML3 cell line shows predominantly cytoplasmic expression of FOXM1 (right panel). OCI-AML3 FOXM1 cells transduced with FOXM1-expressing lentiviral particles show nuclear overexpression of FOXM1. (G) Increased nuclear FOXM1 causes decreased sensitivity of OCI-AML3 cells to chemotherapy agent cytarabine as shown by decreased caspase-3 cleavage. (H) Graph shows quantification as percentage of cell death induced by cytorabine in OCI-AML3 cells with nuclear localization of FOXM1 compared to control cells with cytoplasmic FOXM1, mean+/−SD of a representative triplicate experiments.

FIG. 7, STL427944 treatment causes dose-dependent suppression of FOXM1 protein levels and enhances the cytotoxic effect of conventional chemotherapeutic drugs. [a] Leukemia cells, KG-1, HL-60 and K562 were treated with increasing concentrations of STL427944 for 24 hrs. Total protein samples obtain from treated cells were analyzed for FOXM1 protein levels via immunoblotting, β-actin was used as internal loading control. [b] THP1 and KG-1 cells were treated with indicated concentrations of venetoclax and STL427944 alone or in combination for 24 hrs. [c] KG-1 and K562 cells were treated with indicated concentrations of cytarabine and STL427944 alone or in combination for 24 hrs. In all cases, total protein samples were obtained from cells immediately after treatment and analyzed for Foxm1 and cleaved caspase-3 levels via immunoblotting, β-actin was used as internal loading control.

FIG. 8. STL427944 treatment of human leukemia cell lines suppress FOXM1 protein expression. (a) STL427944 treatment of human leukemia cell lines inhibit FOXM1 protein expression and sensitize cells to venetoclax treatment. (a) STL427944 efficiently reduces FOXM1 protein levels in various human AML cell lines. (b) Stable shRNA-mediated FOXM1 knockdown prominently sensitizes KG-1 cells to cytotoxic effect of venetoclax. (c, d) combined treatment with STL427944 and venetoclax suppresses FOXM1 in KG-1 and THP-1 cells and synergistically promotes cytotoxic effects similarly to FOXM1 knockdown. Total protein was isolated from cells immediately after the treatment and analyzed via immunoblotting with FOXM1 antibodies). Apoptosis induction was estimated based on caspase-3 cleavage.

FIGS. 9A and 9B: Novel FOXM1 inhibitor STL001 sensitizes Leukemia cells to chemotherapy agents. [a] Structural formula of novel FOXM1 inhibitor STL001. [b] Leukemia cells, KG-1, HL-60, and K562 were treated with increasing concentrations of STL001 for 24 hrs. Total protein samples obtain from treated cells were analyzed for FOXM1 protein levels via immunoblotting, β-actin was used as internal loading control. [c] KG-1 cells were treated with indicated concentrations of Laptomycin B, Chloroquine, and STL001 for 24 hrs. Total protein samples obtain from treated cells were analyzed for FOXM1 protein levels via immunoblotting, β-actin was used as internal loading control. [d] KG-1, HL-60, and K562 cells were treated with indicated concentrations of venetoclax and STL001 alone or in combination for 24 hrs. [e] KG-1, HL-60, and K562 cells were treated with indicated concentrations of cytarabine and STL alone or in combination for 24 hrs. In all cases, total protein samples were obtained from cells immediately after treatment and analyzed for Foxm1 and cleaved caspase-3 levels via immunoblotting, β-actin was used as internal loading control.

FIGS. 10A and 10B. STL001 treatment causes dose-dependent suppression of FOXM1 protein levels and sensitize cancer cells of different etiology. [a] Various cell lines representing human triple-negative breast cancer (TNBC), MDA-MB-231, HCC-1143, HCC-1937 were treated with increasing concentration of STL001 for 24 hours. [b] HCC-1143 cells were treated with indicated concentrations of doxorubicin (Dox) and STL001 or in combination for 24 hours. [c] Colorectal cancer cells, HCT-116 and FET were treated with increasing concentration of STL001 for 24 hours. [d] HCT-116 and FET cells were treated with indicated concentrations of 5′ FU (Fluorouracil) and STL001 or in combination for 24 hours. [e] Esophageal cancer cell line, FLO-1 was treated with increasing concentration of STL001 for 24 hours. [f] FLO-1 cells were treated with indicated concentrations of 5′ FU, cisplatin, and STL001 alone or in combination for 24 hours. [g] Ovarian cancer cell lines, ES-2 and OVCAR-8 were treated with increasing concentration of STL427944 and STL001 alone for 24 hours. In all cases, total protein samples were obtained from cells immediately after treatment and analyzed by western blot for FOXM1 and cleaved caspase-3, β-actin was used as internal control.

FIG. 11, reproduced from Bai et al., provides graphs showing FOXM1 expression profile from the TCGA database. The FOXM1 transcript per million are presented in different cancers and corresponding normal tissues, including uterine corpus endometrial carcinoma (a), thyroid carcinoma (b), stomach adenocarcinoma (c), rectum adenocarcinoma (d), prostate adenocarcinoma (e), pheochromocytoma and paraganglioma (f), lung squamous cell carcinoma (g), lung adenocarcinoma (h), kidney renal clear cell carcinoma (i), kidney renal papillary cell carcinoma (j), kidney chromophobe (k), head and neck squamous cell carcinoma (1), glioblastoma multiforme (m), esophageal carcinoma (n), colon carcinoma (o), cholangiocarcinoma (p), cervical squamous carcinoma (q), breast invasive carcinoma (r), liver hepatocellular carcinoma (s), and bladder urothelial carcinoma (t).

FIGS. 12A-12C. FOXM1 is transcriptionally downregulated in NPM1 mutant AML and is an independent predictor of chemotherapy response and disease-specific survival. FIG. 12A Dot and box plot of FOXM1 signature scores (i.e., 1^{st} principal components of RNAseq normalized read counts for 362 genes identified by our prior KG-1 shRNA experiment) by NPM1 status in de novo, FLT3-wildtype AML patients from the Beat AML cohort. The difference in FOXM1 scores by NPM1 group was assessed by the Wilcoxon rank sum test. (n=194 patients). FIG. 12B Overall survival Kaplan-Meier curves for 168 de novo, FLT3-wildtype AML patients treated with standard induction chemotherapy when categorized by NPM1 status and, for NPM1-wildtype patients, FOXM1 transcriptional score (derived from the 362 genes identified from our prior KG-1 shRNA experiment) dichotomized at the sample median. FIG. 12C Multivariable model results for CCR (logistic regression), OS (Cox regression), and disease-related death (Fine-Gray regression). Included Beat AML patients are de novo, FLT3-wildtype, NPM1-wildtype AML treated with standard induction chemotherapy. In the presence of age, sex, leukocytosis and ELN risk score, increased FOXM1 activity remained a potent negative predictor of composite complete remission following chemotherapy (HR 0.37) and inferior survival with a 2-fold increased risk of leukemia-related death (HR=2.02). Abbreviations: n=multivariable model sample size, CCR=Composite Complete Remission, OS=Overall Survival, OR=Odds Ratio, HR=Hazards Ratio, C=Confidence Interval, ELN=European Leukemia Net 2017 prognostic risk classification, univar.=univariable model. Leukocytosis defined as WBC count >11×10⁹/L.

FIGS. 13A-13H STL001 is a novel FOXM1 inhibitor that sensitizes leukemia cells to standard cytotoxic and bcl2 inhibitor therapies. FIG. 13A AML cell line KG-1 treated with precursor compound STL427944. FIG. 3B AML cell lines KG-1, HL-60, and K562 were treated with increasing concentrations of STL001 for 24 h. Total protein samples obtained from treated cells were analyzed for FOXM1 protein levels using immunoblotting; β-actin was used as the internal loading control. FIG. 13C KG-1, HL-60, and K562 cells were treated with indicated concentrations of venetoclax and STL001 alone or in combination for 24 h. FIG. 13D KG-1, HL-60, and K562 cells were treated with indicated concentrations of cytarabine and STL001 alone or in combination for 24 h. In all cases, total protein samples were obtained from cells treatment and analyzed for FOXM1 and cleaved caspase-3 levels via immunoblotting; β-actin was used as internal loading control. FIG. 13E KG-1 cells were treated with indicated concentrations of Leptomycin B, Chloroquine, and STL001 for 24 h. Total protein samples obtained from treated cells were analyzed for FOXM1 protein levels via immunoblotting; β-actin was used as an internal loading control. FIG. 13F KG-1 cell lines with shRNA knockdown of FOXM1 were treated with venetoclax and STL001 and compared to parental cells. FIG. 13G Real-time PCR-based expression profiling for canonical FOXM1 target genes was performed on primary AML mononuclear cells (n=5) treated ex vivo with STL001 5 μM for 24 h. Mean and standard error of the delta Ct for DMSO or treatment group are plotted for each gene. P-value is estimated by two-tailed paired t-test. Asterisks denote P<0.05. FIG. 13H Treatment of peripheral blood mononuclear cells with STL001 for 24 h followed by immunohistochemistry for FOXM1. Slides were scanned on an Aperio AT2 brightfield scanner and images were analyzed using HALO 2.0 software. Hematoxylin counterstain was used to segment nuclei within the ROIs and to establish an accurate cell count. Percent cells with nuclear FOXM1 expression compared between DMSO and STL001 treated cells. P-value is based on one-tailed paired t-test.

FIG. 14 (A-I). Novel FOXM1 inhibitor STL001 causes dose-dependent suppression of FOXM1 protein levels in cancer cell lines of different etiology. (A) Structural formula of novel FOXM1 inhibitor STL001 and its precursor molecule STL427944, modified from source. (B-G) Cancer cells were treated with increasing concentrations of STL427944 (B) and STL001 (C-G) for 24 hrs. Total protein samples obtain from treated cells were analyzed for FOXM 1 protein levels via immunoblotting, β-actin was used as internal loading control (n=3 for each group). (H-I) C3-luc cells stimulated with doxycycline to induce expression of EGFP-FOXM1 fusion protein and FLO-1 cells were treated with increasing concentrations of STL001 for 24 h. Total protein samples were analyzed via immunoblotting for FOXM1 and LC3 expression, β-actin was used as an internal loading control.

FIG. 15. Novel FOXM1 inhibitor STL001 sensitizes esophageal adenocarcinoma cells (FLO-1) to conventional chemotherapeutic drugs through suppression of FOXM1. (A, C, E, G) FLO-1 cells were treated with indicated concentrations of Cisplatin, 5′FU, Paclitaxel, Irinotecan, and STL001 alone or in combination with STL001 for 24 hrs. In all cases, total protein samples were obtained from cells immediately after treatment and analyzed for FOXM1, cleaved caspase-3 levels via immunoblotting, β-actin was used as internal loading control (n=3). (B, D, F, H) Percent (%) dead cells in FLO-1 cells treated with indicated concentrations of Cisplatin, 5′FU, Paclitaxel, Irinotecan, and STL001 alone or in combination with STL001 for 24 hrs. The results shown are the mean±SEM of three independent experiments performed in triplicate (**p<0.001 vs control, n=3). (I and J) FLO-1 cells with stable shRNA-mediated FOXM1 knockdown (KD) were treated with irinotecan (I) and Paclitaxel (J) alone or in combination with STL001 for 24 hrs and compared to parental cells under the same treatment conditions. In all cases, total protein samples were obtained from cells immediately after treatment and analyzed for FOXM1 and cleaved caspase-3 levels via immunoblotting, β-actin was used as internal loading control.

FIG. 16. STL001 enhances the cytotoxic effect of conventional chemotherapeutic drugs in ovarian cancer and colon cancer through suppression of FOXM1. (A) Ovarian cancer (OVCAR-8 and ES-2) cells were treated with indicated concentrations of Doxorubicin (Doxo) alone or in combination with STL001 for 24 hrs. (B) OVCAR-8 cells with stable shRNA-mediated FOXM1 knockdown (KD) were treated with Doxo alone or in combination with STL001 for 24 hrs and compared to parental cells under the same treatment conditions. In all cases, total protein samples were obtained from cells immediately after treatment and analyzed for FOXM1, cleaved caspase-3 levels via immunoblotting, β-actin was used as internal loading control (n=3). (C) colon cancer (HCT-116 and FET) cells were treated with indicated concentrations of 5′FU alone or in combination with STL001 for 24 hrs. (D) HCT-116 cells with stable shRNA-mediated FOXM1 knockdown (KD) were treated with 5′FU alone or in combination with STL001 for 24 hrs and compared to parental cells under the same treatment conditions. In all cases, total protein samples were obtained from cells immediately after treatment and analyzed for FOXM1, cleaved caspase-3 levels via immunoblotting, β-actin was used as internal loading control (n=3).

FIG. 17. STL001 enhances the cytotoxic effect of conventional chemotherapeutic drugs in prostate cancer and breast cancer through suppression of FOXM1. (A) Prostate cancer (22RV1 and LNCaP) cells were treated with indicated concentrations of paclitaxel alone or in combination with STL001 for 24 hrs. (B) Tamoxifen resistance MCF-7 breast cancer cells (TAM-R) were treated with indicated concentrations of Tamoxifen alone or in combination with STL001 for 24 hrs. In all cases, total protein samples were obtained from cells immediately after treatment and analyzed for FOXM1, cleaved caspase-3 levels via immunoblotting, β-actin was used as internal loading control (n=3). (C) Percent (%) dead cells in TAM-R cells treated with indicated concentrations of Tamoxifen alone or in combination with STL001 for 24 hrs. The results shown are the mean±SEM of three independent experiments performed in triplicate (**p<0.001 vs control, n=3). (D-E) Triple negative breast cancer cells (HCC 143) were treated with indicated concentrations of doxorubicin (Doxo) (D) and cisplatin (E) alone or in combination with STL001 for 24 hrs. (F) HCC 143 cells with stable shRNA-mediated FOXM1 knockdown (KD) were treated with doxorubicin alone or in combination with STL001 for 24 hrs and compared to parental cells under the same treatment conditions. In all cases, total protein samples were obtained from cells immediately after treatment and analyzed for FOXM1, cleaved caspase-3 levels via immunoblotting, β-actin was used as internal loading control (n=3).

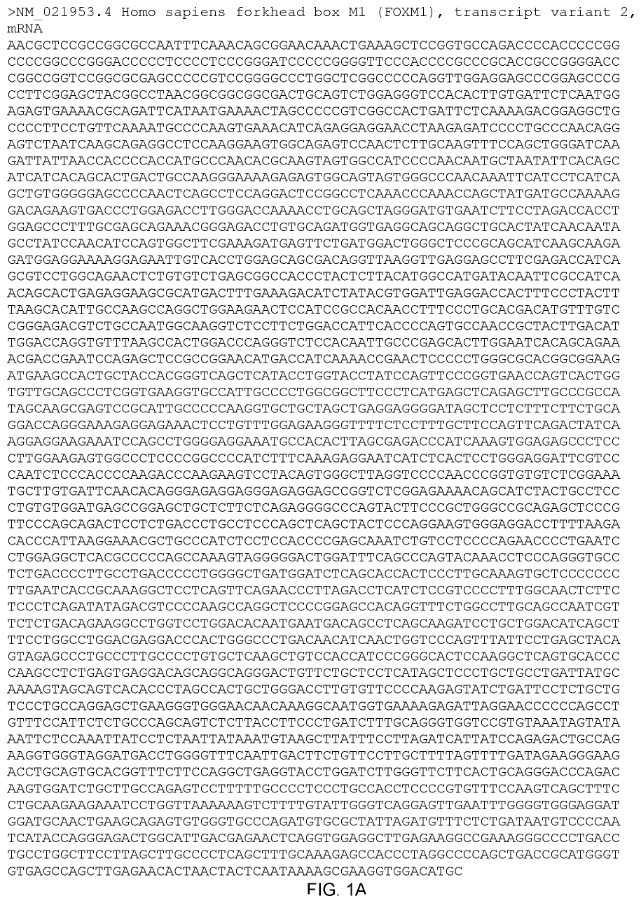

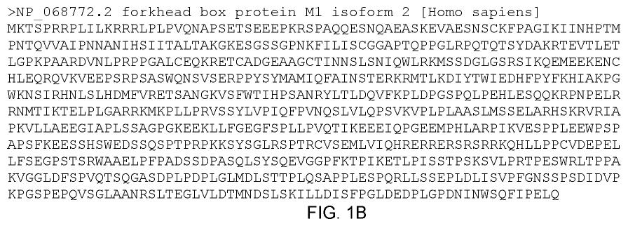

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include, but are not limited to embodiments “consisting essentially of” and “consisting of” these stated elements or steps.

As used herein, the “treatment” or “treating” of a cancer means administration to a patient by any suitable dosage regimen, procedure and/or administration route of a composition, device, or structure with the object of achieving a desirable clinical/medical end-point, including but not limited to, increased survival, reduction or cancer cell number or tumor size, and/or improvement of any other suitable symptom or marker of a cancer. An amount of any reagent or therapeutic agent, administered by any suitable route, effective to treat a patient is an amount capable of treating a cancer. The therapeutically-effective amount of each therapeutic may range from 1 μg per dose to 10 g per dose, including any amount there between, such as, without limitation, 1 ng, 1 μg, 1 mg, 10 mg, 100 mg, or 1 g per dose. The therapeutic agent may be administered by any effective route. For example, in the context of treatment of cancer, may be most typically delivered parenterally or intratumorally, as a primary therapy, or as an adjuvant therapy. The therapeutic agent may be administered as a single dose, at regular or irregular intervals, in amounts and intervals as dictated by any clinical parameter of a patient, or continuously.

Active ingredients, such as the compounds described herein, may be compounded or otherwise manufactured into a suitable composition for use, such as a pharmaceutical dosage form or drug product in which the compound is an active ingredient. Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: anti-adherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts. In one example, a nucleic acid is delivered in a lipid nanoparticle.

Useful dosage forms include: intravenous, intramuscular, intraocular, or intraperitoneal solutions, oral tablets or liquids, topical ointments or creams, and transdermal devices (e.g., patches). In one embodiment, the compound is an intravenous liquid or emulsion.

Suitable dosage forms may include single-dose, or multiple-dose vials or other containers, such as medical syringes or IV bags, containing a composition comprising an active ingredient useful for treatment of cancer.

Pharmaceutical formulations adapted for administration include aqueous and non-aqueous sterile solutions which may contain, for example and without limitation, anti-oxidants, buffers, bacteriostats, lipids, liposomes, lipid nanoparticles, emulsifiers, suspending agents, and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous solutions and suspensions may be prepared from sterile powders, granules, and tablets.

Therapeutic compositions typically must be sterile and stable under the conditions of manufacture and storage. For example, sterile injectable solutions can be prepared by incorporating the active agent in the required amount in an appropriate solvent with one or a combination of ingredients enumerated herein, as required, followed by filter sterilization or other compatible forms of sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe—at least to the extent the benefits of treatment outweighs the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). For example, a single dose or bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., be proportionally reduced or increased as indicated by the exigencies of the therapeutic situation. In some instances, it may be especially advantageous to formulate compositions in dosage unit form for ease of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

The therapeutic agent may be compounded as an adjuvant therapy, with a second therapeutic agent, such as a chemotherapeutic agent. The compound may be provided in the same solution as the second therapeutic agent, such as a chemotherapeutic agent, or may be compounded or packaged separately, such as in a solution in a single vessel, such as an IV bag, or separately with the therapeutic agent in a first vessel, such as an IV bag, and the second therapeutic agent, such as a chemotherapeutic agent, in a second vessel, such as an IV bag, packaged and/or delivered together.

Pharmaceutically acceptable salts, such as acid and base addition salts, are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, (e.g. hydrochloric or hydrobromic acid, sulfuric, nitric, phosphoric and the like acids); or organic acids such as, for example, acetic, propanoic, hydroxyacetic, lactic, pyruvic, oxalic (e.g., ethanedioic), malonic, succinic (e.g., butanedioic acid), maleic, fumaric, malic (e.g., hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely the salt forms can be converted by treatment with an appropriate base into the free base form.

Compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, (e.g. the lithium, sodium, potassium, magnesium, calcium salts and the like), salts with organic bases, (e.g. the benzathine, N-methyl-D-glucamine, hydrabamine salts), and salts with amino acids such as, for example, arginine, lysine and the like. The term “addition salt” as used hereinabove also comprises the solvates which the compounds described herein are able to form. Such solvates are for example hydrates, alcoholates and the like.

The term “quaternary amine” as used hereinbefore defines quaternary ammonium salts which the compounds are able to form by reaction between a basic nitrogen of a compound and an appropriate quaternizing agent, such as, for example, an optionally substituted alkylhalide, arylhalide or arylalkylhalide, (e.g. methyliodide or benzyliodide). Other reactants with good leaving groups may also be used, such as alkyl trifluoromethanesulfonates, alkyl methanesulfonates, and alkyl p-toluenesulfonates. A quaternary amine has a positively charged nitrogen.

Pharmaceutically acceptable counterions include chloro, bromo, iodo, trifluoroacetate, and acetate. The counterion of choice can be introduced using ion exchange resins.

As used herein, unless indicated otherwise, for instance in a structure, all compounds and/or structures described herein comprise all possible stereoisomers, individually or mixtures thereof. The compound and/or structure may be an enantiopure preparation consisting essentially of an (−) or (+) enantiomer of the compound, or may be a mixture of enantiomers in either equal (racemic) or unequal proportions.

A chemotherapeutic agent is any active agent useful for treating cancer. Non-limiting examples of chemotherapeutic agents include: abiraterone acetate, altretamine, amsacrine, anhydro vinblastine, auristatin, bafetinib, bexarotene, bicalutamide, BMS 184476, 2,3,4,5,6-pentafluoro-N-(3-fluoro-4-methoxyphenyl)benzene sulfonamide, bleomycin, bosutinib, busulfan, cachectin, cemadotin, chlorambucil, cyclophosphamide, 3′,4′-didehydro-4′-deoxy-8′-norvin-caleukoblastine, docetaxol, doxetaxel, carboplatin, carmustine (BCNU), chlorambucil, cisplatin, cryptophycin, cyclophosphamide, cytarabine, dacarbazine (DTIC), dactinomycin, daunorubicin, decitabine dolastatin, doxorubicin (adriamycin), etoposide, etoposide phosphate, 5-fluorouracil, finasteride, flutamide, gemcitabine, hydroxyurea, hydroxyureataxanes, ifosfamide, imatinib, irinotecan, liarozole, lonidamine, lomustine (CCNU), Enzalutamide, mechlorethamine (nitrogen mustard), melphalan, mitoxantrone, mivobulin isethionate, rhizoxin, sertenef, streptozocin, mitomycin, methotrexate, nilotinib, nilutamide, onapristone, oxaliplatin, paclitaxel, ponatinib, prednimustine, procarbazine, stramustine phosphate, tamoxifen, tasonermin, taxol, teniposide, topotecan, tretinoin, venetoclax, vinblastine, vincristine, vindesine sulfate, vinflunine, or pharmaceutically acceptable salts or esters thereof. Certain chemotherapeutics may be more suited to certain cancer types, for example and without limitation solid tumors may be treated with doxorubicin, cisplatin, etoposide, or fluorouracil and leukemias may be treated with cytosine arabinoside (cytarabine) or venetoclax.

As used herein, “alkyl” refers to straight, branched chain, or cyclic hydrocarbon groups including, for example, from 1 to about 20 carbon atoms, for example and without limitation C₁₋₃, C₁₋₆, C₁₋₁₀ groups, for example and without limitation, straight, branched chain alkyl groups such as methyl (Me), ethyl (Et), propyl (Pr, including isopropyl, iPr), butyl, pentyl, hexyl, heptyl, octyl, nonyl, decyl, undecyl, dodecyl, and the like. “Substituted alkyl” refers to alkyl substituted at 1 or more, e.g., 1, 2, 3, 4, 5, or 6 positions, which substituents are attached at any available atom to produce a stable compound, with substitution as described herein. “Optionally substituted alkyl” refers to alkyl or substituted alkyl. “Halogen,” “halide,” and “halo” refers to —F, —Cl, —Br, and/or —I. “Alkylene” and “substituted alkylene” refer to divalent alkyl and divalent substituted alkyl, respectively, including, without limitation, methylene, ethylene, trimethylene, tetramethylene, pentamethylene, hexamethylene, hepamethylene, octamethylene, nonamethylene, or decamethylene. “Optionally substituted alkylene” refers to alkylene or substituted alkylene.

“Heteroatom” refers to N, O, P and S. Compounds that contain N or S atoms can be optionally oxidized to the corresponding N-oxide, sulfoxide or sulfone compounds. “Hetero-substituted” refers to an organic compound in any embodiment described herein in which one or more carbon atoms are substituted with N, O, P or S. Use of the prefix “(hetero)” refers to optionally hetero-substituted, for example “(hetero)alkyl” refers to heteroalkyl and alkyl.

“Aryl,” or “Ar”, alone or in combination refers to an aromatic ring system such as phenyl or naphthyl. “Aryl” also includes aromatic ring systems that are optionally fused with a cycloalkyl ring. A “substituted aryl” is an aryl that is independently substituted with one or more substituents attached at any available atom to produce a stable compound, wherein the substituents are as described herein. “Optionally substituted aryl” refers to aryl or substituted aryl. “Arylene” denotes divalent aryl, and “substituted arylene” refers to divalent substituted aryl. “Optionally substituted arylene” refers to arylene or substituted arylene. As used herein, the term “polycyclic aryl group” and related terms, such as “polycyclic aromatic group” means a group composed of at least two fused aromatic rings. “Heteroaryl” or “hetero-substituted aryl” refers to an aryl group substituted with one or more heteroatoms, such as N, O, P, and/or S. “(Hetero)aryl” refers to aryl or heteroaryl.

“Cycloalkyl” refer to monocyclic, bicyclic, tricyclic, or polycyclic, 3- to 14-membered ring systems, which are either saturated, unsaturated, or aromatic. The cycloalkyl group may be attached via any atom. Cycloalkyl also contemplates fused rings wherein the cycloalkyl is fused to an aryl or hetroaryl ring. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein below. “Cycloalkylene” refers to divalent cycloalkyl. The term “optionally substituted cycloalkylene” refers to cycloalkylene that is substituted with 1, 2 or 3 substituents, attached at any available atom to produce a stable compound, wherein the substituents are as described herein.

Cancers include any type of cancer, such as, for example and without limitation, cancers involving solid tumors or leukemias. Due to their a number of factors, for example their tissue origin, their stage or degree of metastases, and their specific genetic and phenotypic makeup, cancers may take innumerable forms, and affect multiple organs or systems. Unless specified, a cancer is treated at any suitable stage or classification. Cancers may be staged using any suitable staging system, such as the TNM staging system or in stages 0, I, II, III, or IV, according to any useful practice and/or standard. Leukemias may be staged differently as compared to cancers involving solid tumors. Cancers may be classified or further classified according to genetic or phenotypic markers, such as by increased or decreased expression of one or more specific genes, or by the presence of specific mutations. Methods of classification and staging of cancers are broadly-known. Leukemias include Acute Myeloid Leukemia (AML), such as cytogenically normal-AML, or other leukemias such as Acute lymphocytic leukemia (ALL), Chronic lymphocytic leukemia (CLL), or Chronic myeloid leukemia (CML). Cancers, such as cancers with solid tumors arise from a tumor mass, and include, without limitation, sarcomas, carcinomas, and lymphomas, such as breast cancer, colorectal cancer, esophageal cancer, or ovarian cancer. Cancers amenable to the treatment methods described herein generally express FoxM1, typically elevated FoxM1 such as nuclear FoxM1 expression, determined, for example by reverse transcriptase quantitative PCR (RT-qPCR), among other methods.

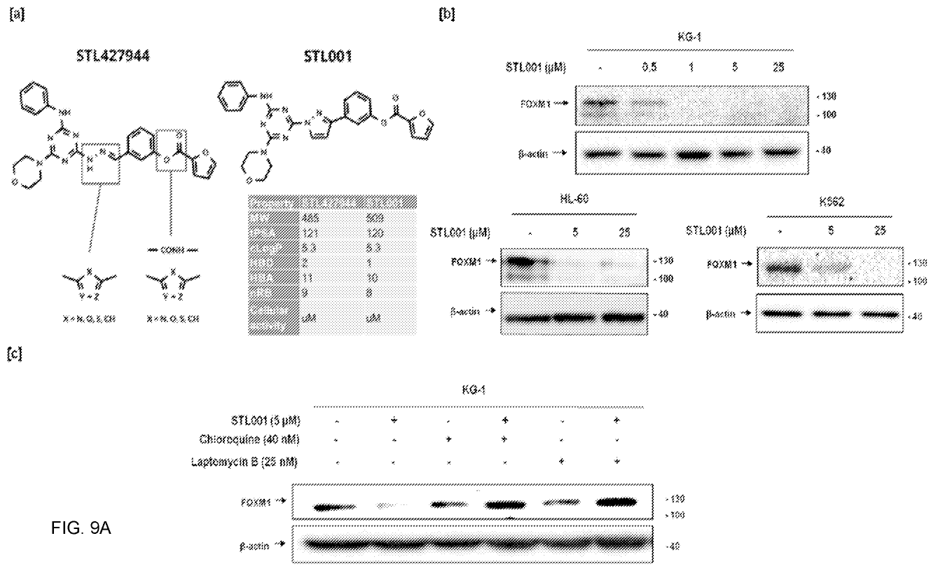

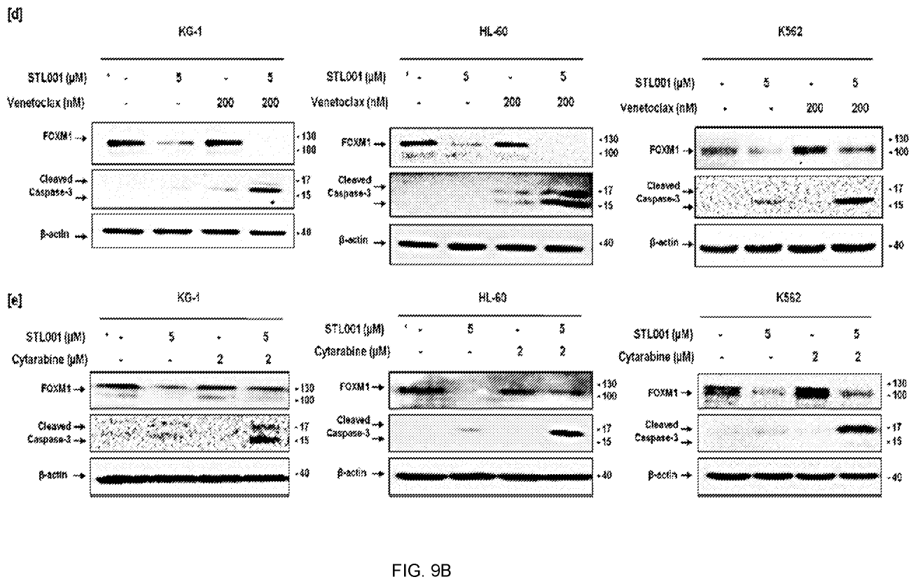

Adjuvant therapies for treating cancer is provided comprising administering a chemotherapeutic agent with a FOXM1 inhibitor compound. The FOXM1 inhibitor compound may be administered by any effective method, e.g., parenterally, for example as an infusion or i.v. dosage form with the chemotherapeutic. The chemotherapeutic may be administered in amounts and dosages effective, and optionally approved for chemotherapies for cancer. The cancer cells may be overexpression FOXM1, e.g. in their nuclei. The FOXM1 inhibitor compound may be STL427944 (see, e.g., FIG. 9A).

The FOXM1 inhibitor compound may have the following structure:

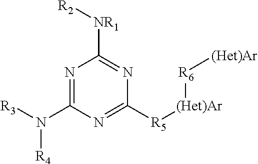

wherein,
(Het)Ar is (hetero)aryl;
R₁ is: H or alkyl, e.g., C₁₋₆ alkyl or C₁₋₃ alkyl;
R₂ is: optionally substituted (hetero)aryl;
R₃ and R₄ are independently: H, Me, Et, cyclopropyl, or R₃ and R₄ taken together are:
   —CH₂CH₂—O—CH₂CH₂—, —CH₂CH₂—N(H)—CH₂CH₂—, —CH₂CH₂—S—CH₂CH₂—, —CH₂CH₂—N(Me)-CH₂CH₂—, —CH₂CH₂—N(Et)-CH₂CH₂—, —CH₂CH₂—S(O)—CH₂CH₂—, —CH₂CH₂—S(O)₂—CH₂CH₂—, —CH₂CH₂—N(H)—CH₂CH₂CH₂—, or —CH₂CH₂—N(Me)-CH₂CH₂CH₂—; and
R₅ and R₆ are, independently, saturated or unsaturated (hetero)cyclopentyl having the structure

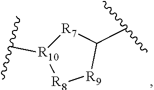

where R₇, R₈, R₉, and R₁₀ are, independently, C, O, N, or S, optionally comprising one or two double bonds in the (hetero)cyclopentyl ring;
   or
      an amide bond, e.g.,

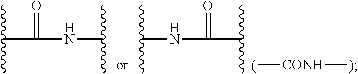

or
an ester bond, e.g.

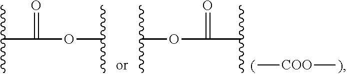

or a pharmaceutically-acceptable salt thereof, including stereoisomers thereof and mixtures of stereoisomers thereof. R₂ may be substituted phenyl, thiophenyl, oxazolyl, thiazolyl, isothiazolyl, furanoyl. R₂ may be substituted with one or more of: —H, -D, -Me, —CHD₂, —CD₃, CH₂D, —F, —CF₃, —Cl, —CN, -Et, -iPr, or —OMe, or a combination of any of the preceding. R₁ may be H.

The FOXM1 inhibitor compound may have the following structure:

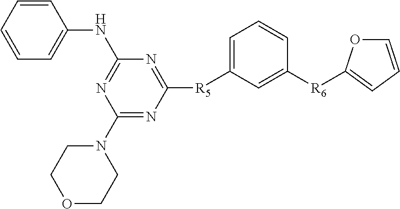

wherein,
R₅ is:
   saturated or unsaturated cyclopentyl or heterocyclopentyl having the structure

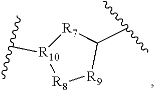

where R₇, R₈, R₉, and R₁₀ are, independently, C, O, N, or S; and
R₆ is:
   saturated or unsaturated cyclopentyl or heterocyclopentyl having the structure

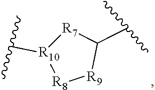

here R₇, R₈, R₉, and R₁₀ are, independently, C, O, N, or S;
   an amide bond; or
   an ester bond,
      or a pharmaceutically-acceptable salt thereof, including stereoisomers thereof and mixtures of stereoisomers thereof.

R₅ may be

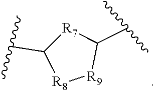

At least one of R₇, R₈, R₉, and R₁₀ may be N, S, or O.

R₆ may be

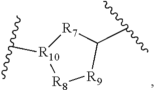

with at least one of R₇, R₈, R₉, and R₁₀ are N, S, or O. R₆ may be

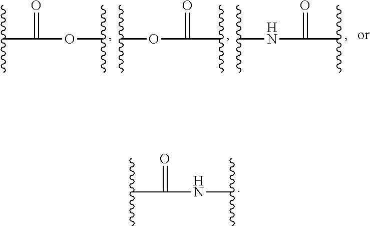

R₅ may be a pyrazole ring, e.g.,

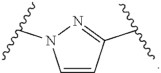 [SMILES: *c1ccn(*)n1]

R₅ may be a pyrrole, furan, thiophene, pyrazole, imidazole, oxazole, isooxazole, pyrrolidine, thiolane (tetrahydrothiophene), or tetrahydrofuran ring.

The FOXM1 inhibitor compound may be STL001, e.g., having the structure:

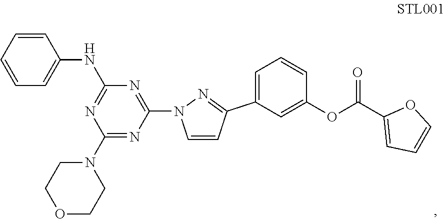 [SMILES: O=C(Oc1cccc(-c2ccn(-c3nc(Nc4ccccc4)nc(N4CCOCC4)n3)n2)c1)c1ccco1]

or a pharmaceutically-acceptable salt thereof, including stereoisomers thereof and mixtures of stereoisomers thereof.

A composition also is provided that may comprise the FOXM1 inhibitor compound according to any embodiment depicted above, and a pharmaceutically-acceptable excipient or carrier. The composition may be in the form of a parenteral dosage form, e.g. an intravenous (iv) product.

The following examples are illustrative, providing description related to the compounds, compounds, and methods described herein, and are not intended to be limiting.

### EXAMPLES

A network-centric transcriptomic analysis was performed to identify a novel commercially available compound STL427944 that selectively suppresses FOXM1 by inducing the relocalization of nuclear FOXM1 protein to the cytoplasm and promoting its subsequent degradation by autophagosomes (see, Chesnokov, M. S. et al. Cell Death and Disease (2021) 12:704). Human cancer cells treated with STL427944 exhibit increased sensitivity to cytotoxic effects of conventional chemotherapeutic treatments (platinum-based agents, 5-fluorouracil, and taxanes). RNA-seq analysis of STL427944-induced gene expression changes revealed prominent suppression of gene signatures characteristic for FOXM1 and its downstream targets but no significant changes in other important regulatory pathways, thereby suggesting high selectivity of STL427944 toward the FOXM1 pathway. Collectively, the novel autophagy-dependent mode of FOXM1 suppression by STL427944 validates a unique pathway to overcome tumor chemoresistance and improve the efficacy of treatment with conventional cancer drugs. Medicinal chemistry optimization of this compound achieved a potent and selective novel (No—IP) compound with improved drug-like properties and therapeutic potential. Although STL427944 is promising, as described herein, modification of the structure of STL427944 provides superior efficacy.

### Example 1—Chemical Synthesis

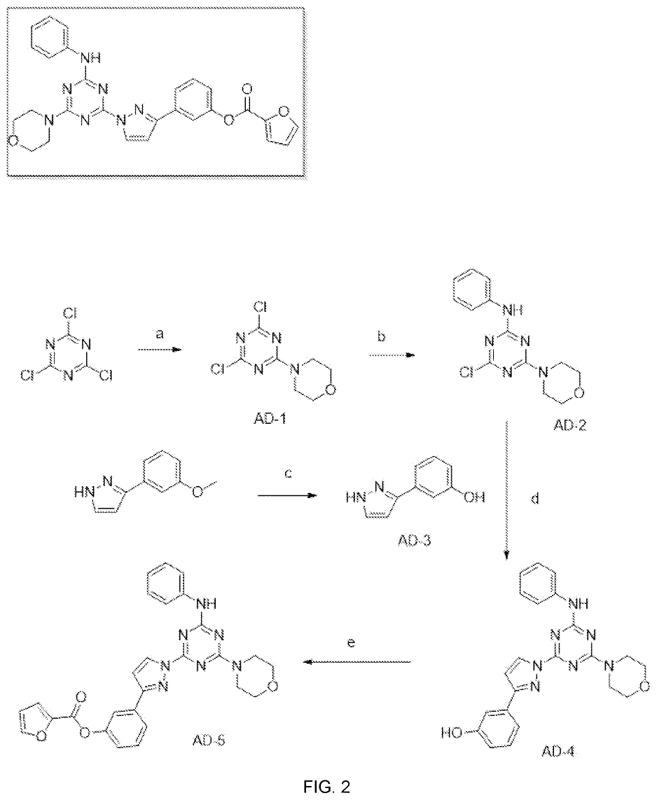

Synthesis of the compound STL001 (AD-5) is shown in FIG. 2, details follow.

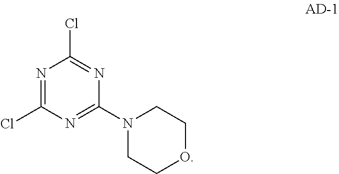 [SMILES: Clc1nc(Cl)nc(N2CCOCC2)n1]

To a stirred solution of cyanuric chloride (5.0 g, 27 mmol) in acetone (50 ml) was added dropwise a solution of morpholine (1.7 g, 19 mmol) and triethylamine (1.9 g, 19 mmol) in acetone (50 ml) at −20° C. for 3 hours. The reaction was monitored with TLC. The resulting mixture was quenched with water, filtered, washed with water and cold methanol and dried to give product. Yield: 70%.

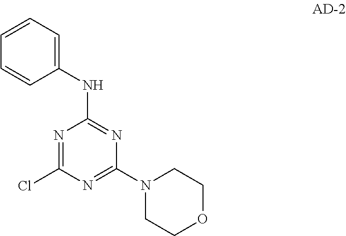 [SMILES: Clc1nc(Nc2ccccc2)nc(N2CCOCC2)n1]

To a stirred solution of AD-1 (460 mg, 2 mmol) in THE 3 mL, aminobenzene (2 mmol, 2 mmol) was added, then K₂CO₃ (6 mmol) was added. The reaction mixture was held at rt for 4 h and then removed K₂CO₃ by filtration and removed THE by evaporation. The residue was extracted by water and DCM. The organic phase was evaporated to dryness and run column by diethyl ether and petroleum ether (5%). Yield: 72%.

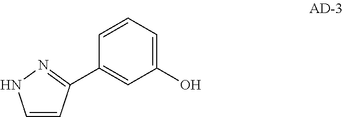 [SMILES: Oc1cccc(-c2cc[nH]n2)c1]

To a solution of 3-(3-methoxyphenyl)-IH-pyrazole (0.51 g, 3 mmol) in DCM (30 mL) was cooled down to −78° C., treated with boron tribromide (30 mmol). The mixture was stirred at −78° C. for 3 h. Then, the reaction mixture was warmed up to room temperature and stirred at room temperature for overnight. The mixture was quenched with H₂O (10 mL), extracted with EtOAc (30 mL×3). The combined organic phase was dried over sodium sulfate. The organic phase was concentrated to give a crude residue. The crude product was purified with column chromatography with DCM/MeOH to get the pure product. Yield: 60%.

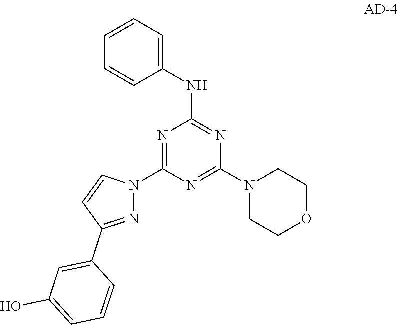 [SMILES: Oc1cccc(-c2ccn(-c3nc(Nc4ccccc4)nc(N4CCOCC4)n3)n2)c1]

To a stirred solution of AD-3 (320 mg, 2 mmol, 1.0 eq) and AD-2 (580 mg, 2 mmol, 1.0 eq) in DMF, K₂CO₃ (6 mmol) was added. The reaction mixture was heated to 110° C. for overnight. The reaction was monitored with TLC. The reaction was quenched with brine, and was extracted by ethyl acetate. The organic phase was evaporated to dryness and the crude residue was purified by column chromatography to get the pure compound. Yield: 65%.

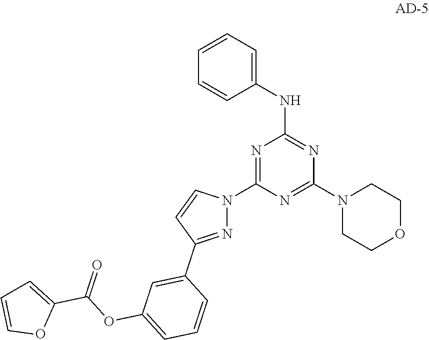 [SMILES: O=C(Oc1cccc(-c2ccn(-c3nc(Nc4ccccc4)nc(N4CCOCC4)n3)n2)c1)c1ccco1]

To a stirred solution of AD-4 (415 mg, 1 mmol, 1.0 eq) and furan-2-carboxylic acid (112 mg, 1 mmol, 1.0 eq) in THE (6 mL), EDC-HCl (229 mg, 1.2 mmol, 1.2 eq), DMAP (37 mg, 0.20 mmol) and Et₃N (2.0 eq) were added. The reaction mixture was stirred at room temperature for overnight. The reaction was monitored with TLC. The reaction was quenched with brine, and was extracted by ethyl acetate. The organic phase was evaporated to dryness and the crude residue was purified by column chromatography to get the pure compound. Yield: 73%.

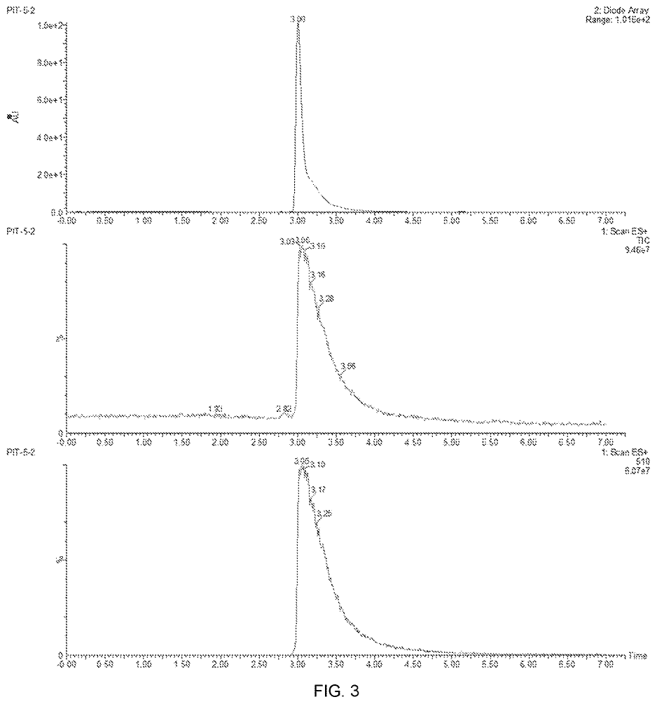

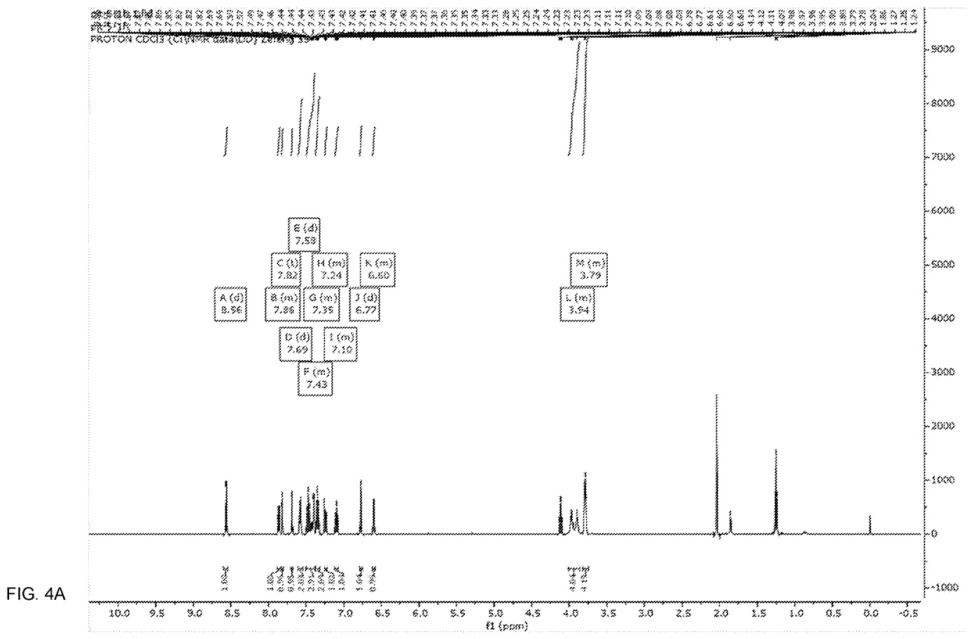

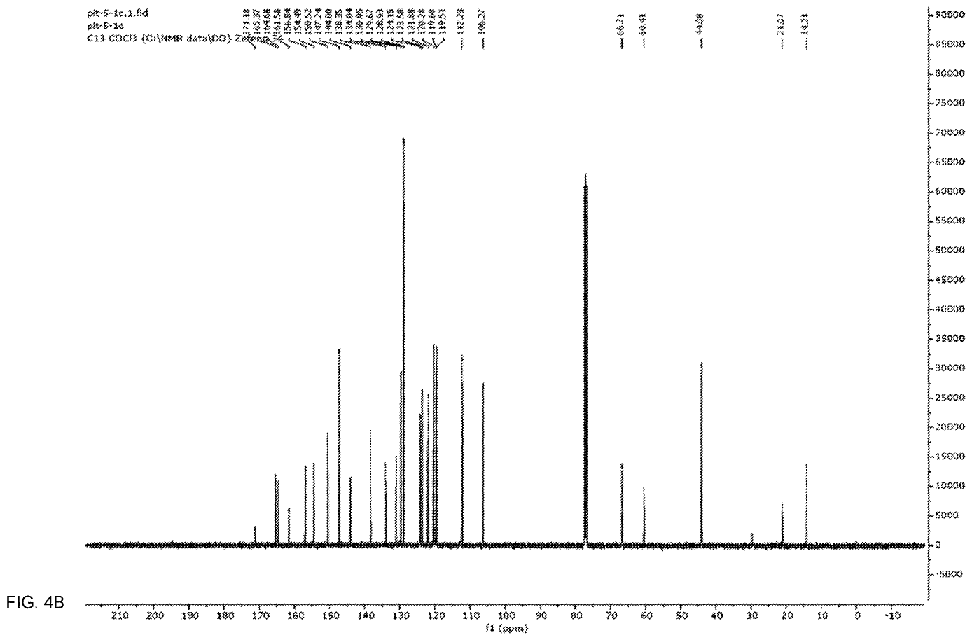

AD-5, formula C₂₇H₂₃N₇O₄, exact mass 509.1812 (FIG. 3): H-NMR: ¹H NMR (500 MHz, Chloroform-d) δ 8.56 (d, J=2.8 Hz, 1H), 7.88-7.85 (m, 1H), 7.82 (t, J=2.0 Hz, 1H), 7.69 (d, J=1.6 Hz, 1H), 7.58 (d, J=8.0 Hz, 2H), 7.50-7.38 (m, 3H), 7.37-7.32 (m, 2H), 7.25-7.22 (m, 1H), 7.12-7.07 (m, 1H), 6.77 (d, J=2.8 Hz, 1H), 6.62-6.58 (m, 1H), 4.01-3.86 (m, 4H), 3.82-3.74 (m, 4H). C-NMR: ¹³C NMR (126 MHz, CDCl₃) δ 171.18, 165.37, 164.68, 161.58, 156.84, 154.49, 150.52, 147.24, 144.00, 138.35, 134.04, 130.95, 129.67, 128.93, 124.15, 123.58, 121.88, 120.28, 119.68, 119.51, 112.23, 106.27, 66.71, 60.41, 44.08, 21.07, 14.21 (FIGS. 4A and 4B).

### Example 2

NPM1 mutations are the most common mutations in CN-AML (Cytogenetically Normal-AML). FOXM1 is one of the most over-expressed genes in many human cancers including AML. In addition, the FOXM1 regulatory network is a major predictor of poor prognosis in human cancers of different origin, including AML. FOXM1 is involved in all hallmarks of cancer and targeting this transcription factor may lead to the inhibition of cancer development. In AML cells, nuclear WT-NPM protein determines nuclear localization of FOXM1, while mutant NPM sequesters FOXM1 in the cytoplasm. It is shown herein that a FOXM1 specific inhibitory compounds, e.g, STL001 and STL427944, suppresses the expression of FOXM1 in AML cell lines and sensitizes AML cells to cytarabine and venetoclax. Strikingly, similar to what is seen in CN-AML, STL001 also localizes FOXM1 to the cytoplasm. Improved outcome for AML patients with FLT3-WT and mutant NPM1 is linked to the cytoplasmic localization, and consequent functional inactivation, of FOXM1 as a transcription factor in the cytoplasm, suggesting that nuclear FOXM1 induces chemoresistance of AML.

In parallel, STL001 is evaluated in combination with standard chemotherapy in mouse models of AML, with the ultimate goal of establishing that the mode of action for a favorable outcome for patients with mutant NPM1 is the inactivation of the transcription factor FOXM1 by preventing its nuclear translocation. From a therapeutic point of view, the studies are expected to unveil the mechanism by which FOXM1 promotes chemoresistance and will suggest broader strategies for targeting nuclear FOXM1 for treatment of AML.

This considers a novel molecular mechanism as an explanation of favorable prognosis for patients with FLT3-WT-AML/mutant NPM1. This mechanism may explain the resistance and sensitivity of AML patients to chemotherapy. NPM1 is mutated in 30% of all AML cases, but the mutation occurs more frequently (40% to 60%) in CN-AML resulting in its nuclear export and a more favorable prognosis. In studies of AML patients with WT FLT3 (FLT3-WT AML), those with NPM1 mutations showed superior overall survival and relapse-free survival. Moreover, it has been shown for these patients that chemotherapy alone is sufficient for treatment. However, the underlying mechanism to explain this phenomenon is lacking. The model described herein provides novel paradigm in which improved prognosis is linked to the cytoplasmic localization and functional inactivation of FOXM1 in FLT3-WT AML cells, with mutant NPM1 resulting in the inactivation of the FOXM1 regulatory network.

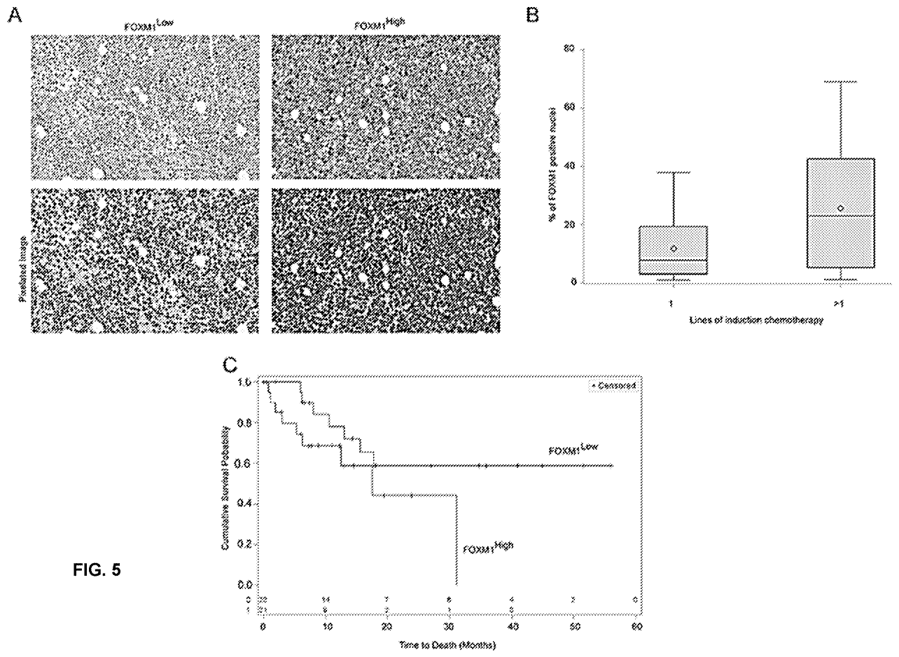

A paradigm shift in AML treatment for patients with wild-type FLT3/NPM1 is provided—to target nuclear FOXM1 in these cells. In preliminary experiments, we showed that patients with nuclear FOXM1 exhibit significantly reduced survival profiles (FIG. 5), and the efficacy of chemotherapy in NPM1^{wt}-AML cell lines was increased after inhibition of nuclear FOXM1 by RNAi. In contrast, overexpression of exogenous FOXM1 in the nucleus of NPM1^{mut} cells with cytoplasmic localization of FOXM1 resulted in increased resistance of these cells to chemotherapy. These data suggest that nuclear FOXM1 is responsible for the worse outcome for AML patients with NPM1^{wt}. Conversely, the observation that FOXM1 inactivation leads to favorable prognosis provides fertile ground for strategies to inhibit this oncogenic transcription factor in AML.

FOXM1 inhibits HOXA expression in AML cells, while knock-down of FOXM1 leads to the activation of the HOXA locus and to sensitivity to anti-cancer drugs. We will determine the role of HOXA activation in AML chemo-sensitization.

We identified a novel compound, STL001 that acts as the specific FOXM1 inhibitor in cancer cells. Identification of novel therapeutic strategies for the treatment of AML is extremely important. Inhibiting transcription factors is still an open problem in medicinal chemistry. Hence, we developed and applied a highly innovative network-centric approach to identify and optimize a first-in-class degrader of FOXM1. FOXM1 is overexpressed in the majority of AML cases, and FOXM1 expression correlates with resistance to treatment and poor survival for AML patients.

Acute Myeloid Leukemia (AML) is a highly heterogeneous disease with a 3-year survival ranging from 30-80% in patients depending on its molecular characteristics. Mutations in nucleophosmin (NPM1) occur in about one third of the AML cases in adults, making it one of the most common mutations seen in this disease. Patients with this distinct type of AML typically have a normal karyotype, a mutant NPM1 gene (NPM1^{mut}), and an excellent response to induction chemotherapy, but the mechanistic basis for this improved prognosis was not known. The NPM1 gene encodes a nucleocytoplasmic shuttle protein that is localized to the nucleolus under steady state conditions. Sequencing the coding region of NPM1 in leukemic blasts revealed all mutations to be localized to exon 12, where they result in the mis-localization of NPM to the cytoplasm.

In subsequent studies of patients with AML who had a normal karyotype, those with mutated NPM showed superior overall survival (OS) and relapse-free survival (RFS). However, about 40% of NPM1-mutated patients also acquire an internal tandem duplication in fms-like tyrosine kinase (FLT3) and are classified as FLT3-ITD-positive. The FLT3-ITD creates an in-frame transcript, which is translated into a protein that is constitutively active and promotes ligand-independent proliferation and survival of leukemic blasts, leading to an unfavorable outcome even in the background of mutant NPM1. The basis for this outcome is unknown.

A recent meta-analysis of over 1900 patients under age 60 with known NPM1 mutation status confirmed a prognostic advantage with mutated NPM1, yielding a hazard ratio (HR) of 0.56 for OS, and 0.37 for RFS. Moreover, a retrospective analysis of AML patients, some of whom underwent a stem cell transplant, showed that the presence of an NPM1 mutation obviated the benefit of this aggressive therapy leading to a strong clinical recommendation (category IA) to treat these patients with chemotherapy alone. In spite of the remarkable progress achieved in biologic, diagnostic, and clinical characterization of NPM mutations since their first identification in early 2005, the mechanism by which the NPM1 mutant contributes to favorable prognosis remained elusive.

Since we showed that NPM mutant protein drives FOXM1 into the cytoplasm and renders FOXM1 inactive in tumor cells, we explored the role of nuclear FOXM1 as a novel marker for risk stratification and potential driver of AML. Our retrospective study examined 74 samples of adults with intermediate risk AML. Clinical data was collected in parallel with bone marrow biopsy slides that were stained with FOXM1 antibody and DAPI. Expression of FOXM1 was quantified utilizing the Aperio ScanScope and Digital Imaging analysis suite (FIG. 5 (A)). All patients included in the study were treated with intensive chemotherapy at diagnosis and treatment response classified by International Working Group criteria. The patients needing >1 line of therapy to achieve remission had a higher percentage of nuclei expressing FOXM1 (FIG. 5 (B)) at diagnosis, suggesting its importance as a predictor of chemoresistance (mean 25.6% vs. 11.4% FOXM1 positive nuclei, 2-tailed t-test: p=0.004). The samples from both institutions were analyzed individually for overall survival due to institutional variation in post-induction treatment strategies. In 43 AML patients from a single institution in this dataset, high FOXM1 average nuclear intensity is predictive of inferior overall survival (median 501 days vs. not reached, p=0.07) (FIG. 5 (C)) when patients are stratified based on median FOXM1 nuclear intensity. These collective data suggests that unfavorable outcome for AML patients may be linked to higher expression of nuclear FOXM1.

Oncogenic role of NPM1c. There are essentially two mechanisms underlying the oncogenic effects of the NPM1c mutation. The first is the loss of wild type NPM1 from the nucleus where it may exert important tumor suppressor effects. The p14 (Arf) tumor suppressor is exported from the nucleolus where it is unstable and degraded. Loss of nuclear Arf by NPM1c inhibits its interaction with MDM2 and blunts the p53 response. In addition, proteomic studies demonstrate that mutant NPM1 results in cytoplasmic export of PU.1, a transcription factors that serves as a member of the PU.1/CEBPA/RUNX1 transcription factor complex that is critical for granulomonocytic differentiation. However this observation was not borne out in human samples. The second effect and one that is has more recently been elucidated is oncogenic gain function from the cellular re-localization of the protein. Early studies using the CD34+ purified AML patient cells in PDX models resulted in a leukemia phenotype supporting its role as a founder mutation. This is further supported by the distinctive gene expression profile in this patient subset and the absence of other recurrent cytogenetic abnormalities. Moreover, its frequent presence at relapse suggest it is a critical oncogenic driver in AML and make it one of the best touted MRD markers in AML. The oncogenic function of NPM1c is reinforced by the anti-leukemic effects of inhibition of nuclear exports of NPM1c as well as inhibition of oligomerization of NPM. Early zebrafish models show an expansion of hematopoietic stem cells with expression of the cytoplasmic NPM1 mutant protein. Recent conditional knock in models of the human NPM1 mutation indicate enhanced proliferation in the committed myeloid progenitor cells and increased self-renewal capacity that culminates in the gradual development of leukemia. NPM1 mutant AML cells showed upregulates HOX expression via inactivation of FOXM1. Apparently FOXM1 negatively regulates expression of HOXA locus. HOX overexpression is critical to maintaining the LSC state. Removal of the NPM1c mutant using CRISPR-Cas9 gene elegantly resulted in downregulation of HOXA expression and restoration of myeloid differentiation. A recent publication highlighted the MLL-menin complex as a therapeutic vulnerability of NPM1 mutated AML and showed an oral inhibitor of the menin-MLL interaction VTP-50469 was able to eradicate NPM1c mutant AML cells in xenografts and prevent leukemia development in the knock-in murine model.

While the bulk of published literature suggest the NPM1 mutation has oncogenic functions in AML, this mutation results in enhanced chemosensitivity in AML, and patients with this mutation are at low risk of relapse. In studies of patients with AML, in the absence of other deleterious mutations, those with NPM1 mutations showed superior overall survival and relapse-free survival. As above, a recent meta-analysis of over 1900 patients under age 60 with known NPM1 mutation status confirmed a prognostic advantage with mutated NPM1 yielding a hazard ratio (HR) of 0.56 for OS, and 0.37 for RFS. Moreover, a retrospective analysis of AML patients some of whom underwent a stem cell transplant in AML, showed that the presence of an NPM1 mutation obviated the benefit of this aggressive therapy. The current WHO and ELN classify these patients as favorable risk AML with a clinical recommendation to treat these patients with chemotherapy alone. However, the underlying mechanism to explain this phenomenon was previously lacking. We postulated that NPM1 mutants exert their effect by binding and dislocating other protein partners into leukemic cell cytoplasm and consequently interfering with their functions. Identifying a putative oncogenic driver complexed to nucleophosmin would provide mechanistic insight into the chemo-sensitivity that characterizes NPM1-mutant AML.

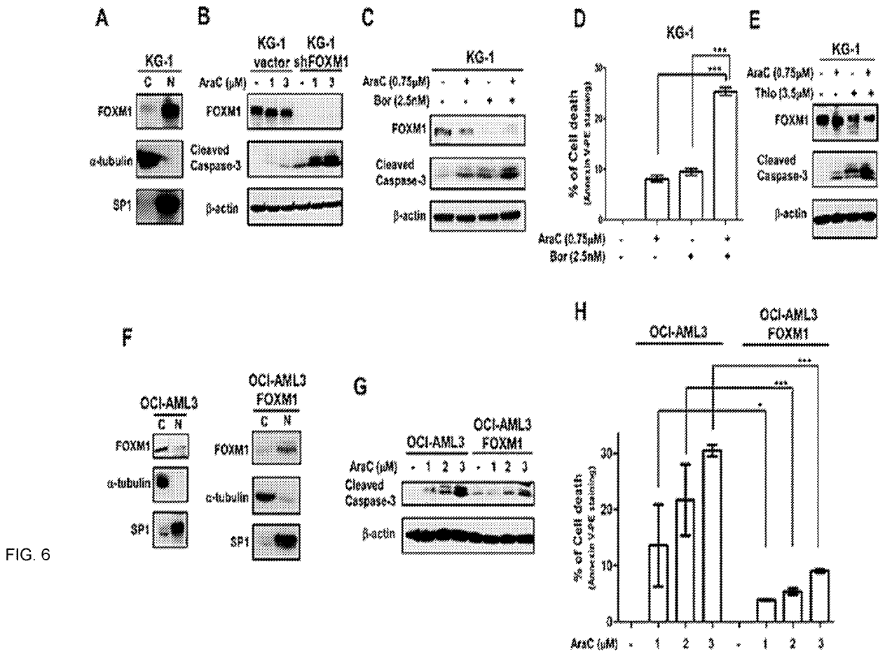

Cell lines recapitulated the observation that FOXM1 localization in AML is contingent on NPM1 mutational status. We then tested the role of FOXM1 in mediating chemoresistance in leukemia. We hypothesized that high levels of nuclear FOXM1 may contribute to chemoresistance. OCI-AML3 cells with NPM^{mut} predominantly express cytoplasmic FOXM1. We therefore determined if overexpression of nuclear FOXM1 in this cell line could confer resistance to chemotherapy (See, FIG. 6). OCI-AML3 cells were transduced with lentiviral particles to overexpress FOXM1, and single clones were selected. Cellular fractionation confirmed overexpression of exogenous FOXM1 in the nuclear compartment. After treatment with increasing concentrations of cytarabine for 24 hours, cells overexpressing FOXM1 showed reduced cell death by caspase-3 cleavage in comparison with the parental OCI-AML3 cell. These experiments clearly show the opposing effects of nuclear versus cytosolic FOXM1 on chemoresistance of AML cells, supporting the idea that the favorable outcomes of patients with WT FLT3 and mutant NPM1 are partially attributable to the cytoplasmic inactivation of FOXM1.

Next, we analyzed gene expression changes induced in KG-1 (AML) cells by stable FOXM1 knockdown (“FOXM1-KD” gene-set) using RNA-seq. A total of 10,698 protein-coding genes were significantly affected in either one or both examined samples with 3,578 genes (33.4%) displaying bi-directional changes in both samples. “FOXM1-KD” gene-sets exhibited two clearly discernible groups of common up- and down-regulated genes identified by unsupervised hierarchical clustering. We identified the clusters of overexpressed and repressed genes in “FOXM1-KD” group to determine genes potentially associated with AML clinical phenotype and detected dramatic increases in the expression of multiple genes belonging to HOXA family (HOXA1, HOXA3, HOXA5, HOXA6, HOXA7, HOXA11, HOXA13). Notably, high HOXA expression in AML was recently reported to be associated with NPM1 mutations and increased sensitivity to venetoclax treatment. We therefore evaluated the cytotoxic action of venetoclax in control and FOXM1-deficient KG-1 cells and demonstrated that stable FOXM1 knockdown indeed results in prominent sensitization of AML cells to venetoclax effects. To predict possible regulators and pathways that may be responsible for STL-induced gene expression changes, we performed an integrative analysis of regulatory pathways using Ingenuity software.

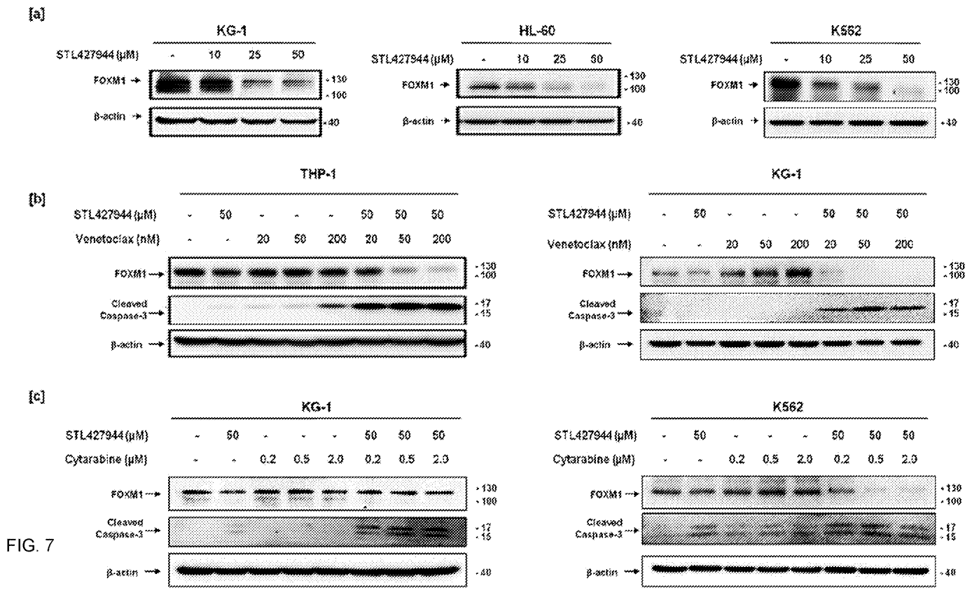

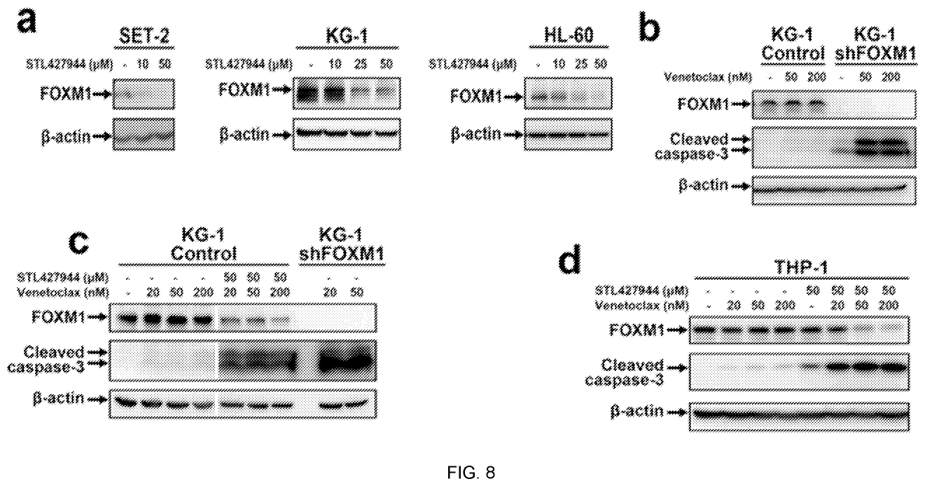

FOXM1 inhibitors, STL427944 and STL001 induce FOXM1 degradation in AML cells. To evaluate the mechanism of FOXM1 inhibition through a network centric approach, we used the LINCS dataset to correlate the differential gene expression profiles of knock-out genes in the transcriptional network of FOXM1 with those of cells treated with compounds in same cancer cell lines. This phenotypic screening elucidated STL427944 C₂₅H₂₃N₇O₄; STL) (PubChem CID #252119181) as a FOXM1 inhibitor, overcoming chemoresistance of several conventional chemotherapeutic treatments (platinum-based agents, 5-fluorouracil, and taxanes) in several cell lines. Potency of this compound ii AML estimated to be in uM range and we demonstrated that it suppresses the expression of FOXM1 in AML at concentrations from 10 μM to 50 μM. Combinations of STL427944 and venetoclax in KG-1/THP-1 cells led to synergetic induction of apoptosis indicated by caspase-3 cleavage, suggesting a potential rationale for combination therapy with these drugs. Moreover, the efficiency of KG-1 sensitization to venetoclax treatment by STL427944 was comparable to effects caused by shRNA-mediated FOXM1 knockdown. See, FIGS. 7 and 8.

For certain applications, however, the STL4527944 in vivo half-life and potency could be limiting. An initial modification of STL427944 (STL001) that increases its metabolic stability was much more effective at suppressing FOXM1 up 0.5 μM in AML cell lines (FIGS. 9A and 9B). From a medicinal chemistry standpoint of view, the compound has two main metabolic liabilities, a hydrolytically labile furane-carboxylic acid phenolic ester and a hydrazone. We applied several paths of structural activity relationship (SAR) optimization to overcome these issues (as shown in FIGS. 9A and 9B), ranging from removing of liabilities to bio-isosteric replacement to cyclizations. Out of ten newly designed compounds STL001 shown in FIGS. 9A and 9B showed up to 50-fold improvement in efficiency to suppress FOXM1. The ring replacement is likely to have significantly improved the metabolic stability of our first hit, resulting in a more cellular active compound, with better ‘drug-like’ properties.

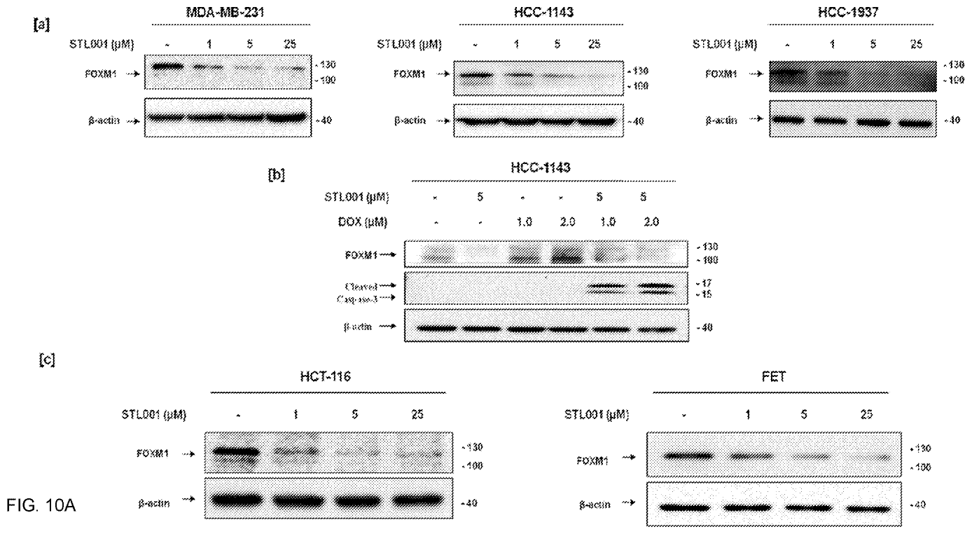

Efficacy in solid tumor cells. Similar to AML cells, STL001 demonstrably lowers FOXM1 expression and sensitivity to chemotherapeutics in solid tumors, such as breast cancer, colorectal cancer, esophageal cancer, and ovarian cancer cells. STL427944 also shows efficacy in knocking down FOXM1 expression in ovarian cancer cells, but with lesser efficacy as compared to STL001. Caspase-3 cleavage indicates increased apoptosis in concurrent treatment with traditional chemotherapeutics such as doxorubicin, cisplatin and fluorouracil. See FIGS. 10A and 10B.

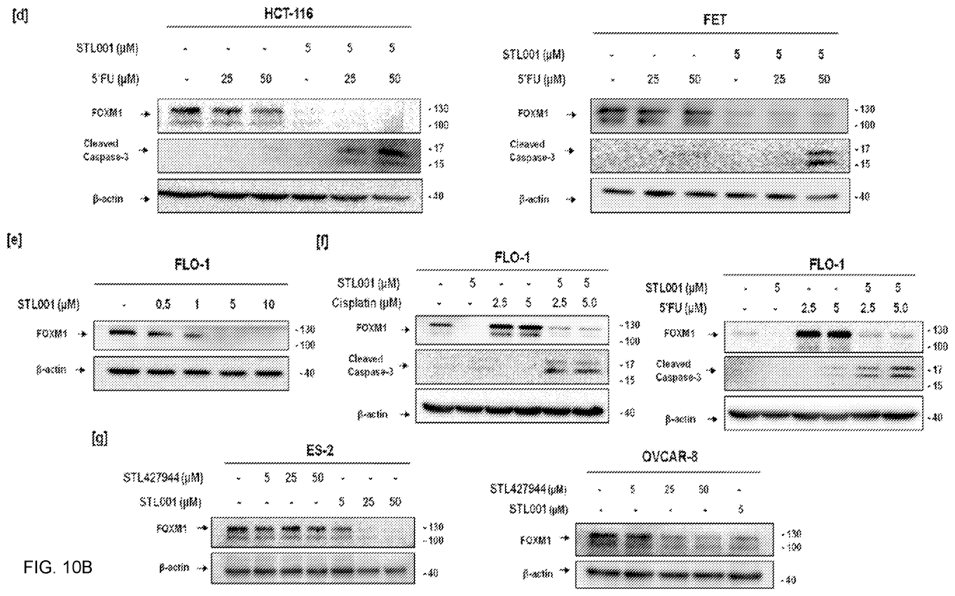

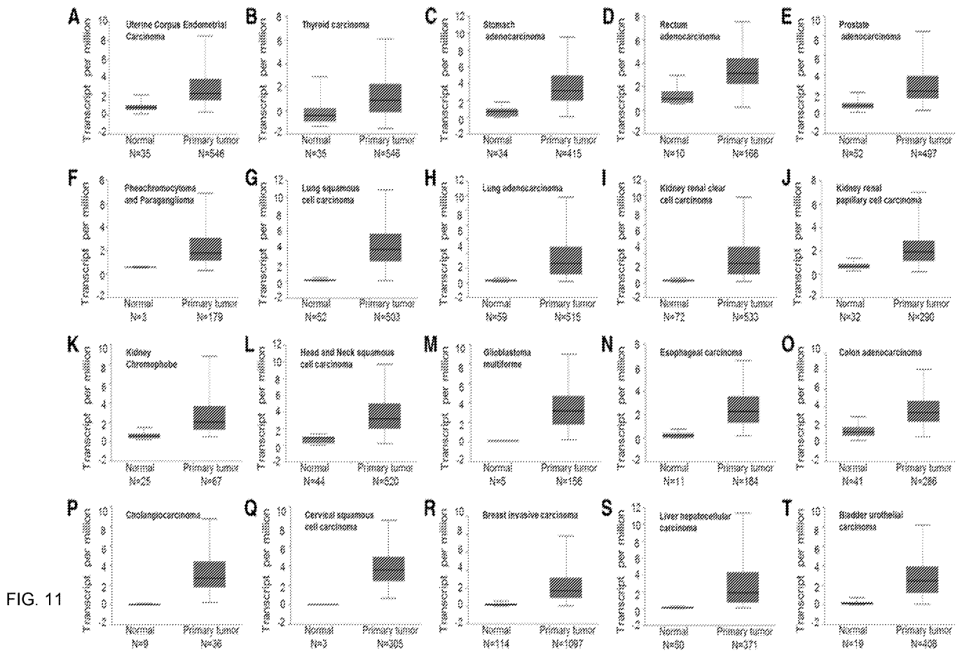

FOXM1 is increased in a variety of human cancers, such as, without limitation: ovarian cancer, breast cancer, prostate cancer, hepatoma, angiosarcoma, colorectal cancer, melanoma, lung cancer, and gastric cancer, consistent with the results obtained from the TCGA database (see, e.g., FIG. 11, reproduced from Bai et al. Cell Commun Signal. 2018 Sep. 12; 16(1):57). Therapeutically-effective amounts of the compounds described herein are expected to be effective adjuvant therapeutics to chemotherapy for the treatment of those cancers.

To predict possible regulators and pathways that may be responsible for STL427944-induced gene expression changes, we performed an integrative analysis of regulatory pathways using Ingenuity software. All predicted signaling changes and outcome effects strongly suggest that STL427944 treatment should exert strong anti-tumor effects. To additionally verify our predictions regarding the impact of STL427944 on regulatory pathways, we performed GSEA for “STL427944 signature” genes using Pathway Interaction Database (PID) collection of gene signatures. Out of 196 signatures analyzed, 6 gene sets were significantly enriched. Moreover, ATR and E2F activity can be modulated by FOXM1, implying that pathways responsible for STL427944-induced gene expression changes converge on FOXM1. Taken together, our transcriptomic data strongly suggest that FOXM1 is a central driver mediating changes in gene expression after STL427944 treatment. STL001 also is expected to act as a specific inhibitor of FOXM1.

### Example 3

RNA-sequencing (RNAseq) of KG-1 cells was performed with stable shRNA-mediated FOXM1 knockdown. Those RNAseq results were used to define a FOXM1 transcriptional signature comprising genes with |log 2 fold change (FC)|>10 when comparing expression in FOXM1-deficient cells to control scramble vector transduced cells. While 407 genes from this KG-1 cell line experiment met the-fold change criterion, 362 of the genes had normalized RNA expression values in Beat AML patients. The 362 genes were examined within a cohort of n=194 de novo AML patents with a wild-type FLT3 (FLT3 wt) genomic profile and available Beat AML RNAseq data. The analysis was restricted to FLT3 wt AML because deleterious FLT3-ITD mutations can activate FOXM1 directly through AKT signaling.

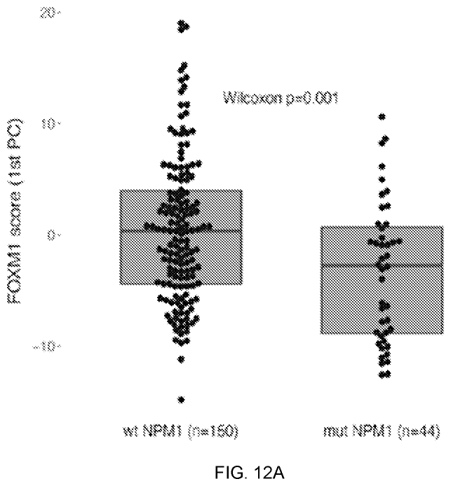

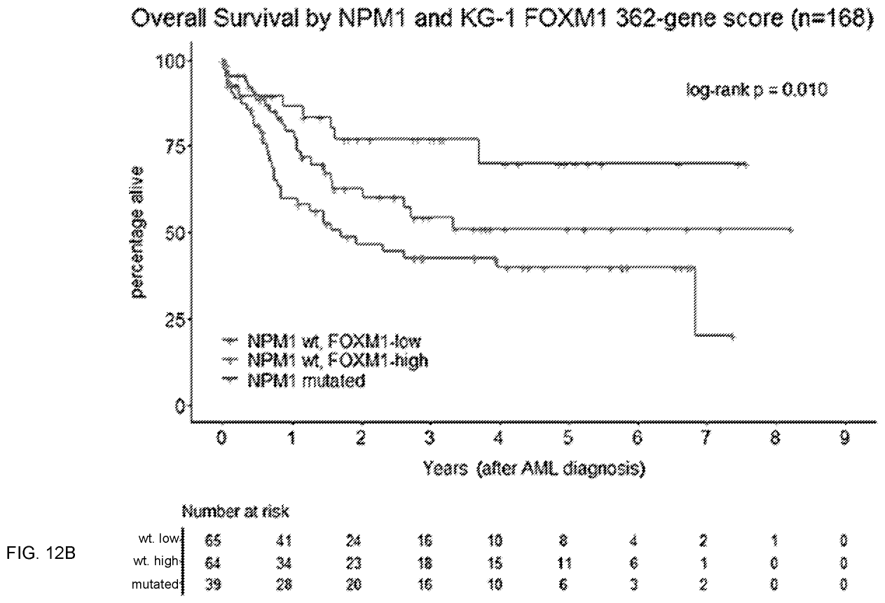

An unclustered heatmap of RNA expression values (conditional quantile normalized and gene [row] standardized) identified the subset of differentially expressed genes between NPM1 mutant (n=44) and NPM1 wild type (n=150) patient samples—defined as NPM1 mut/NPM1 wt |log 2 FC|>1. The heatmap was ordered by NPM1 status fold change with the 93 genes downregulated in NPM1 mut compared to NPM1 wt listed above and the 32 genes upregulated in NPM1 mut AML. The heatmap showed an overall downregulation of FOXM1 activity in NPM1 mutant AML patient samples and likely represents the subset of FOXM1 target genes that contribute to the favorable outcome conferred by the NPM1 mutation. A FOXM1 transcriptional signature score was calculated for each Beat AML patient as the first principal component (i.e., eigengene) of conditional quantile (accounting for library size, GC content, and gene length) and specimen type normalized RNAseq read counts of the previously described 362 genes. These FOXM1 scores were significantly lower in NPM1mut patients compared to NPM1 wt patients (FIG. 12A) demonstrating for the first time that NPM1 mutations are determinants of FOXM1 transcriptional activity.

The prognostic relevance was examined of FOXM1 transcriptional activity limiting the analysis to patients treated with intensive chemotherapy (n=168). Overall survival (OS) following intensive chemotherapy was compared between 3 groups, NPM1 mut AML patients (n=39) and NPM1 wt patients (n=129) stratified into FOXM1-low and FOXM1-high groups based on median FOXM1 score. The Kaplan Meier survival curves demonstrate OS in FOXM1-low (s median) NPM1 wt patients was superior to the FOXM1-high (>median) NPM1 wt patients and approached the survival curve of the favorable subset of NPM1 mut AML patients (3-group log rank test p=0.010) (FIG. 12B). The pairwise OS comparison between the two NPM1 wt groups (high vs. low FOXM1 transcriptional score) had HR=1.55 (95% Cl: 0.92-2.61) and Wald test p=0.097 from a univariable Cox model (data not shown). A multivariable analysis was conducted controlling for well-validated patient and disease characteristics known to correlate with AML prognosis (e.g., age, ELN risk group). A high FOXM1 score was associated with inferior overall survival rate, OS (HR=1.52, p=0.139) and a two-fold increased risk of a disease-related death, disease-specific survival (HR=2.02, p=0.039) among the subgroup of patients with NPM1 wt treated with standard induction chemotherapy. Importantly, again in the multivariable setting, a high FOXM1 score was associated with over 60% reduction in the odds of achieving a composite complete remission after induction therapy among these NPM1 wt patients (OR=0.37, p=0.041) (FIG. 12C). These findings establish that low FOXM1 activity is an independent predictor of chemotherapy response and disease-specific survival for AML patients. The current data represent the first clinical validation that the FOXM1 transcriptional signature is linked to NPM1 mutational status and modulates treatment outcomes thereby making it an attractive target.

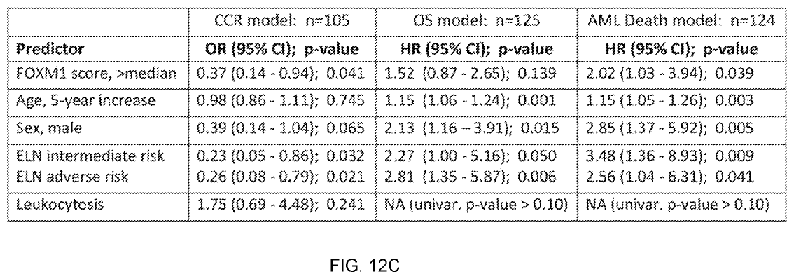

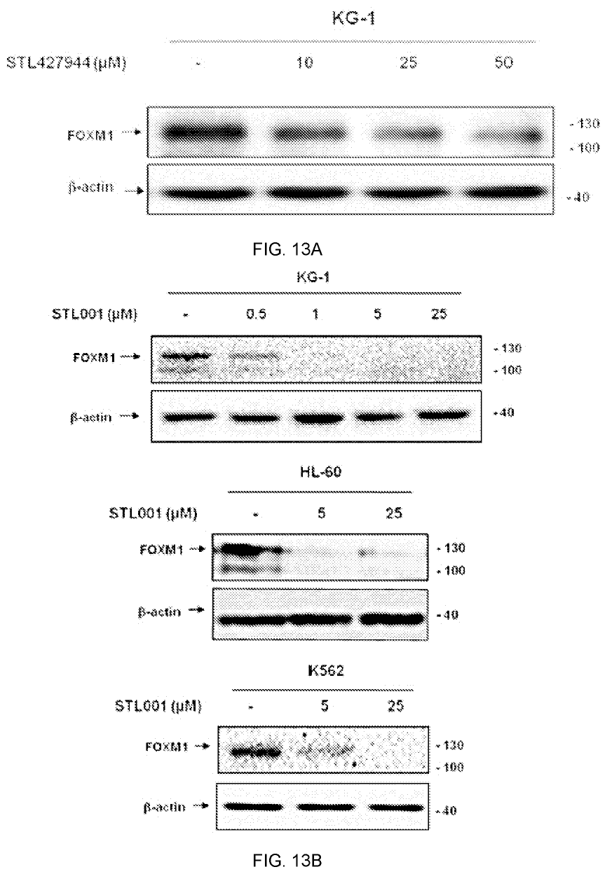

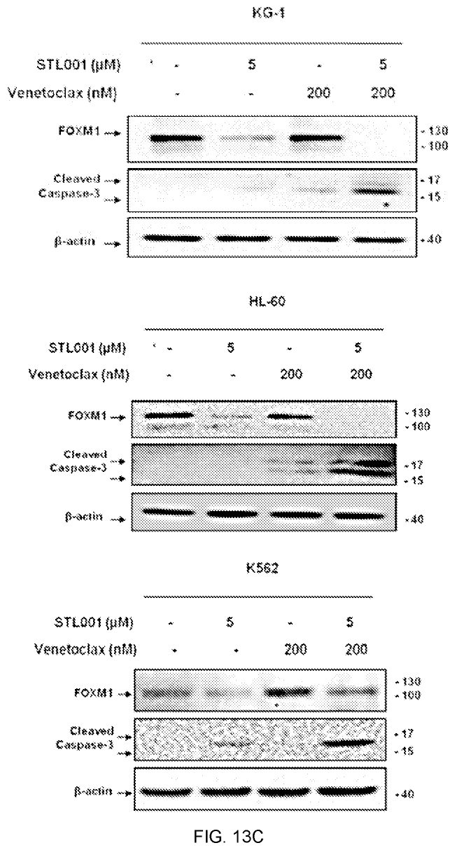

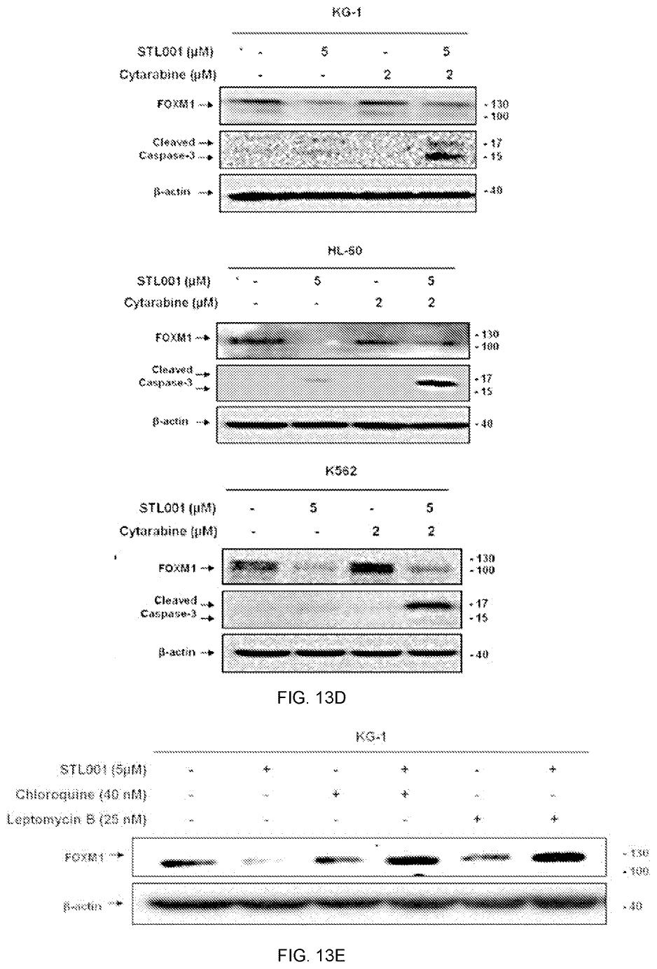

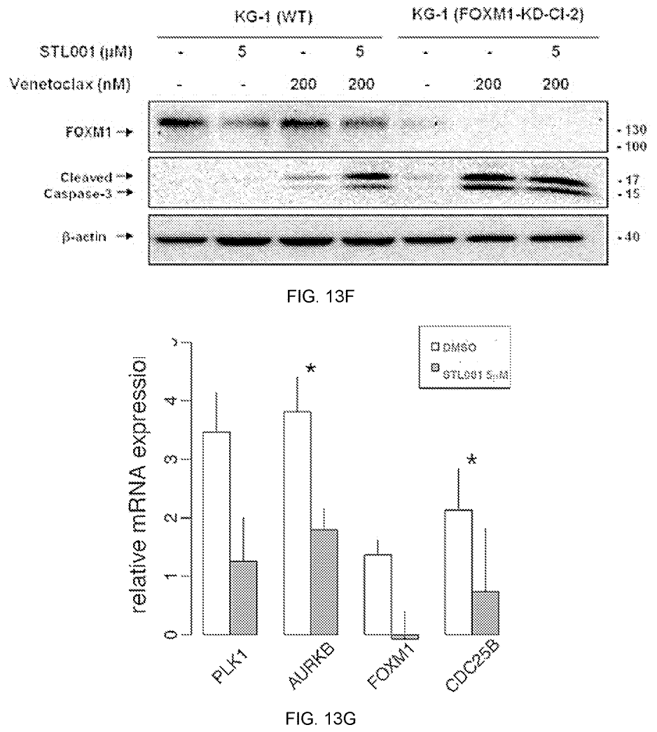

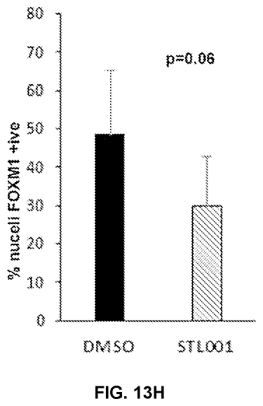

It was postulated that FOXM1 inhibitors will suppress FOXM1 function, thus mimicking the effect of the NPM1 mutation. Based on STL427944, several paths of structural activity relationship optimization were performed and, out of 10 newly designed compounds, STL001 showed up to 50-fold increased potency as a FOXM1 inhibitor. The ring replacement is likely to have significantly improved the metabolic stability of the predecessor molecule, resulting in a more cellular active compound with better ‘drug-like’ properties. Upon treating a panel of AML cell lines—including KG-1, HL-60, and K562—with increasing concentrations of STL001 for 24 h, a dose-dependent inhibition of FOXM1 protein expression was observed at significantly lower concentrations (as low as 500 nM) compared to the precursor compound (STL427944) that showed modest FOXM1 inhibition at concentrations of 25 μM (FIGS. 13A and 13B). STL001 treatment did not exert prominent cytotoxic action on its own, but use in combination with either venetoclax (FIG. 13C) or cytarabine (FIG. 13D) in KG-1, HL-60 and K562 cells led to potent induction of apoptosis indicated by caspase-3 cleavage, providing a rationale for combination therapy with these drugs. The addition of Leptomycin B or Chloroquine rescued FOXM1 protein levels from suppression by STL001 (FIG. 13E), suggesting that STL001 induces cytoplasmic re-localization of FOXM1 and subsequent autophagy-dependent degradation.

These results indicate that the first-generation modification drug, STL001, preserves the mode of action of the parent compound, STL427944, and potentially has the same high selectivity towards FOXM1. Moreover, this treatment sensitization effect of STL001 is absent in KG-1 cells with stable knockdown of FOXM1 (FIG. 13F), suggesting that the key effect of the compound on chemosensitization is through FOXM1 inhibition. These findings were validated in primary AML samples (n=5) where ex vivo treatment with STL001 followed by real-time PCR analysis showed significantly suppressed expression of canonical FOXM1 transcriptional targets AURKB and CDC25B (FIG. 13G) in addition to downregulation of PLK1 and FOXM1 itself, due an autoregulation loop. In addition, primary AML samples (n=4) were treated ex vivo with STL001 and slides were scanned on an Aperio AT2 brightfield scanner and images were analyzed using HALO 2.0 software. The % of nuclei staining positive for FOXM1 was calculated per cell and averaged per slide to analyze FOXM1 protein expression and localization. Results confirmed a decrease in nuclear FOXM1 protein levels after STL001 treatment (FIG. 13H) and approached statistical significance (43% vs. 29% of nuclei expressing FOXM1, paired t-test, one-tailed p=0.06).

Overcoming resistance to chemotherapeutic drugs and BCL2 inhibitors represents a cornerstone issue in improving survival outcomes in AML. While there has been tremendous progress in understanding the oncogenic function of NPM1 mutations in AML, little insight has been made into the fortuitous sensitization to chemotherapy and BCL2 inhibitors conferred by this mutation. The suppressed FOXM1 transcriptional activity in NPM1 mut AML patients in the current work brings to light a potential avenue to uncouple the oncogenic from the chemosensitizing effect of this highly prevalent mutation. Here, it is shown for the first time that FOXM1 transcriptional activity in AML patient samples can determine response to chemotherapy and independently predict disease-related death. Collectively our previous and current data suggest FOXM1 is an actionable target for AML patients with wild-type NPM1. Pharmacologic inhibition of FOXM1 may recapitulate the effects of the NPM1 mutation through the inactivation of FOXM1 and, thereby, confer more favorable treatment outcomes to NPM1-wild type AML patients with a current, dismal median survival of less than 1 year.

### Example 4—FOXM1 Inhibitor STL001 Sensitizes Different Human Cancers to Broad Spectrum of Anticancer Drugs

The capacity of STL001 as a FOXM1 inhibitor was verified in human cancer cell lines from solid tumors. Further, we showed here that FOXM1 inhibitor STL001 treatment resulted in sensitization of cancer cells to apoptotic death by multiple chemotherapeutic agents. STL001 was studied further to verify its direct target engagement with FOXM1. Also provided is Transcriptome-supported evidence that STL001 exhibits selectivity toward suppressing FOXM1-controlled regulatory pathways. This study serves to verifies that STL001, and by extension other compounds described herein, effectively antagonize FOXM1 activity and sensitize a variety of human cancer cells to traditionally used chemotherapy agents, and may be suitable for further clinical evaluation in targeting chemotherapy resistance human cancers.

### Materials and Methods

Cell culture: Human cell lines LNCaP and 22Rv1 (human prostate carcinoma), OVCAR8 and ES-2 (High-grade serous ovarian cancer, HGSOC), HCT-116 and HCT-FET (human colorectal carcinoma), HCC-1143 (triple negative breast cancer (TNBC), TAMR (Tamoxifen resistant MCF7) human breast cancer, were provided from various investigators. LNCaP and 22Rv1 cell lines were cultured in RPMI-1640 with 2 mM L-Glutamine (Gibco; Thermo Fisher Scientific, Waltham, MA, USA). HCC-1143 cell lines were cultured in Iscove's Modified Dulbecco Medium (IMDM) with 2 mM L-Glutamine (Gibco; Thermo Fisher Scientific). OVCAR-8, ES-2, HCT-116, and HCT-FET cell lines were cultured in Dulbecco's Modified Eagle Medium (DMEM) with 4.5 g/L glucose and 4 mM L-Glutamine (Gibco; Thermo Fisher Scientific). For all cell lines, the growth media was supplemented with 10% Foetal Bovine Serum (FBS), penicillin (100 U/mL), and 100 μg/mL streptomycin (Gibco; Thermo Fisher Scientific). TAMR cells were routinely cultured in DMEM/F12 medium without phenol red (Gibco; Thermo Fisher Scientific), containing 1% charcoal-striped FBS, 2.5 mM L-Glutamine (Thermo Fisher Scientific), 6 ng/mL insulin (Millipore Sigma) and 50 nM 4-hydroxytamoxifen (4-OHT; Millipore Sigma). All cell lines were grown and maintained at 37° C. in a humidified incubator with 5% CO₂. Sub-confluent cultures (70-80%) were split 1:5 using 0.25% Trypsin/EDTA (Millipore Sigma).

Cells were confirmed to be mycoplasma-free by routine testing using PCR based tests and DAPI-staining with subsequent evaluation by biological fluorescence microscopy.

Chemical compounds and drugs: STL001, Cisplatin (AdipoGen Life Sciences, USA), Paclitaxel (APExBIO Technology, USA), 5-FU (LKT Laboratories, USA), and Doxorubicin (Thermo Fisher Scientific) were dissolved in DMSO (Millipore Sigma). Tamoxifen (Millipore Sigma) was dissolved in ethanol. Puromycin (Millipore Sigma) was dissolved in sterile water. Estrogen, β-Estradiol (E2) was provided dissolved in ethanol.

Drug treatment of cultured cells: After harvesting, cells were counted in presence of Trypan Blue (Thermo Fisher Scientific) and seeded onto tissue culture plates to achieve ~50% confluency. The next day, treatment of cells was performed by aspirating the nonadherent cells and growth medium and replacing it with the fresh one containing selected concentrations of drugs. For Tamoxifen based studies, the nonadherent cells and growth media was aspirated and replaced with the DMEM supplemented with 4.5 g/L glucose and 4 mM L-Glutamine, 10% FBS, penicillin (100 U/mL), 100 μg/mL streptomycin, 10 nM estrogen, and selected concentrations of drugs. The vehicle control groups were treated using the solvent of the drug, vehicle concentration did not exceed 0.3%. Post-treatment at selected times, the cells were harvested, washed with ice-cold PBS and used for protein or RNA purification as described below.

Stable FOXM1-expression knockdown in cancer cell: Cells were seeded on commercially available 12-well tissue culture plates to achieve ~40% confluency. The next day, cells were transduced with MISSION© lentiviral particles carrying pLKO.1 vector encoding a non-targeting shRNA control or shRNA against human FOXM1 transcripts (Millipore Sigma) at multiplicity of infection (MOI) 10 in the presence of Polybrene (10 μg/mL) and allowed to incubate for 24 h at 37° C. in a humidified incubator with 5% CO2. Transduced cells were selected by their cultivation with puromycin (1.0 μg/mL) for 10 days and then maintained without puromycin as described above.

Protein immunoblotting: Total protein was extracted using ice-cold radio-immunoprecipitation assay (RIPA) buffer (Millipore Sigma) supplemented with Halt protease- and phosphatise-inhibitor cocktails (Fisher Scientific), 2 mM sodium orthovanadate (New England Biolabs, Inc., USA), and 5 mM sodium fluoride (Millipore Sigma) according to the manufacturer's protocol. Protein content in each sample was estimated using Bio-Rad Protein Assay (Bio-Rad, USA). Equal amounts of protein (20-30 μg) were separated on hand-cast SDS/PAGE (6-12%) mini-protein gels and transferred to 0.2 μm Immobilon-Psq polyvinylidene difluoride (PVDF) transfer membrane (Millipore Sigma). Membranes were blocked with 4% bovine serum albumin (BSA; Millipore Sigma) in TRIS-buffered saline (TBS) with 0.1% Tween-20 (TBS-T, Thermo Fisher Scientific) and probed overnight at 4° C. with the primary antibodies (FOXM1, Cell Signaling Technology (CST), Inc., USA; Cleaved Caspase-3, CST; β-actin, Thermo Fisher Scientific) diluted according to the manufacturer's protocol. When appropriate, the membranes were then washed with TBST for 15 min and proved with the HRP-conjugated secondary antibodies for 2 hrs at room temperature. After incubating membranes with the secondary antibodies, the membranes were then washed three times for 10 min each with TBST. Protein bands were developed using SuperSignal™ West Pico PLUS Chemiluminescent Substrate (Thermo Fisher Scientific) and detected using ChemiDoc Imaging System (Bio-Rad). The molecular weights of protein makers are indicated on the right of each immunoblot image in the various FIGS.

Full-transcriptome RNA-seq: Total RNA from cultured cells was extracted and purified using TRIzol reagent (Fisher Scientific) and the PureLink™ RNA Mini Kit (Fisher Scientific) including on-column DNase (Thermo Fisher Scientific) treatment according to the manufacturer's instructions. To assess the integrity of RNA, all samples were analyzed on the Agilent 4200 TapeStation (Agilent Technologies, USA). The remaining DNA concentrations were measured using the Qubit fluorometer (Thermo Fisher Scientific). In all the samples the DNA amounts did not exceed 2% of the total amount of nucleic acid.

Sequencing libraries for Illumina sequencing platform were created in one batch in a 96-well plate, using Stranded CORALL Total RNA-Seq Library Prep kit (Lexogen, Austria) with Lexogen's RiboCop HMR rRNA Depletion Kit. In brief, in the first step during rRNA removal we used 260-660 ngs of total RNA, and then followed by library creation initiated with random oligonucleotide primer hybridization with complementary sequence within the RNA template and reverse transcription. No prior RNA fragmentation was done before reverse transcription, as the insert size was determined by proprietary size-restricting method. Next, Illumina-compatible P5 sequences and UMIs (Unique Molecular Identifiers) were ligated at the 3′ end of the first-strand cDNA fragments. During the following steps of the 2^{nd}-strand cDNA synthesis and the double-stranded cDNA amplification, unique i5 and i7 index sequences as well as complete adapter sequences required for cluster generation were added. The number of PCR amplification cycles was 12, as determined by qPCR using a small pre-amplification library aliquot for each sample.

Subsequently, the final PCR amplified libraries were purified and quantified. Finally, prior to sequencing average fragment sizes confirmed to be 325 bp by Agilent 4200 TapeStation (Agilent Technologies). The final library pool concentration was confirmed by qPCR and then subjected to test sequencing in order to check sequencing efficiencies and adjust accordingly the proportions of individual libraries. Sequencing was carried out on the Illumina NovaSeq 6000 system with S4 flow cell (Illumina, USA), approximately 30 μM 2×150-bp clusters per sample.

Bioinformatical Analysis of RNA-Seq Data: Sequencing data were aligned to human reference genome version GRCh38 annotated by Gencode version 43, using STAR (Dobin A, et al. STAR: ultrafast universal RNA-seq aligner. Bioinformatics. 2013 Jan. 1; 29(1):15-21). Counts within genes were obtained by Feature Counts (Liao Y, et al. featureCounts: an efficient general purpose program for assigning sequence reads to genomic features. Bioinformatics. 2014 Apr. 1; 30(7):923-30). Differential expression in STL001 versus control and in FOXM1-KD versus control for autosomal protein-coding genes was assessed by the likelihood ratio test, based on negative binomial distribution as implemented in DESeq2 (Love M I, et al. Moderated estimation of fold change and dispersion for RNA-seq data with DESeq2. Genome Biol. 2014; 15(12):550). Nominal P-values were adjusted for multiple comparisons using Benjamini and Hochberg approach (Benjamini Y, Hochberg Y. Controlling the false discovery rate: a practical and powerful approach to multiple testing. Journal of the Royal Statistical Society Series B (Methodological) 1995; 57:289-300). Significant genes were determined by adjusted P-value<0.05 and fold changes lower than 0.5 or higher than 2.0. NIH DAVID (Dennis G Jr, et al. DAVID: Database for Annotation, Visualization, and Integrated Discovery. Genome Biol. 2003; 4(5):P3. Epub 2003 Apr. 3) was used for gene enrichment in Gene Ontology biological processes (Ashburner M, et al. Gene ontology: tool for the unification of biology. The Gene Ontology Consortium. Nat Genet. 2000 May; 25(1):25-9). Gene set enrichment analysis (Subramanian A, et al. Gene set enrichment analysis: a knowledge-based approach for interpreting genome-wide expression profiles. Proc Natl Acad Sci USA. 2005 Oct. 25; 102(43):15545-50) in Pathway Interaction Database (PID) collection of curated and peer-reviewed canonical pathway gene signatures used the Preranked algorithm with a number of permutation set to 1000. Pathways with FDR<0.01 were considered significant.

### Results

STL001 decreased FOXM1 protein expression levels in human cancer cells: STL427944, reduces chemoresistance of cancer cells by inducing FOXM1 degradation (Chesnokov, M. S., et al. Novel FOXM1 inhibitor identified via gene network analysis induces autophagic FOXM1 degradation to overcome chemoresistance of human cancer cells. Cell Death Dis 12, 704 (2021)). STL427944 was identified by transcriptomic network analysis and confirmed in various cancer cell lines as a selective inhibitor of FOXM1 at very high concentrations (Id.). However, from a medicinal chemistry perspective, the compound STL427944 has metabolic liabilities. To overcome these issues; STL001 was developed, as described above, showing up to 50-fold estimated increase in potency as FOXM1 inhibitor. STL001 is a new molecule with similar biological properties to the parent compound STL427944, however the ring replacement in the parental compound is likely to have significantly improved the overall stability and better drug-like’ properties and thus enhanced potency observed in STL001. To assess experimentally the FOXM1-suppressing effect of the novel compound STL001, we used a panel of human solid cancer cell lines with high FOXM1 expression levels, including ovarian cancer (OVCAR-8, ES-2), colorectal cancer (HCT-116, HCT-FET), breast cancer (TAM-R, HCC-1143), and prostate cancers (22RV1, LNCaP). All cell lines were treated with STL001 (1, 5, and 10 μM) resulted in a dose-dependent reduction of the cellular levels of FOXM1 protein at significantly lower concentrations (1 μM). Consistent with our results with AML, the present results demonstrate that STL001 is a universal inhibitor of FOXM1 in cancer cells, however it is ~25 times more efficient in reducing the cellular FOXM1 activity in solid cancer cell lines as compared to its parental compound STL427944, which shows modest FOXM1 suppression at concentrations of 25-50 μM [Id.].

STL001-induced FOXM1 suppression in sensitized cancer cells to chemotherapeutic agents: Chemoresistance is a major barrier for the traditionally used anti-cancer drugs and FOXM1 over expression is closely associated with chemoresistance and poor survival in most solid tumors, whereas FOXM1 down regulation is proved very effective in restoring chemotherapy sensitivity in several cancer cells. Considering FOXM1 as a regulator of sensitivity and resistance in cancer cells, we assumed that STL001 treatment induced FOXM1 suppression should reduce chemoresistance and sensitize cancer cells to cytotoxic effects of chemotherapies.

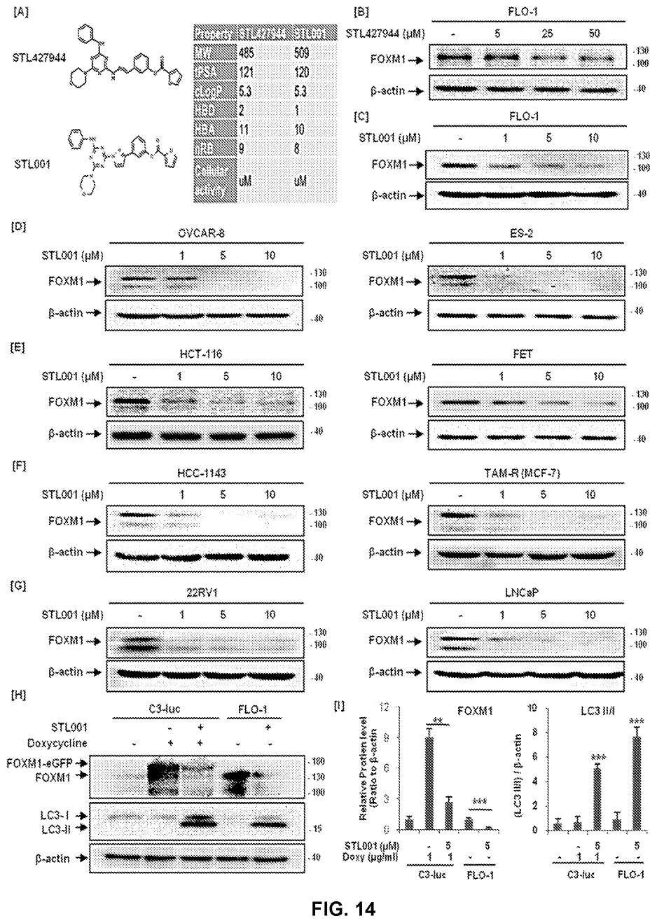

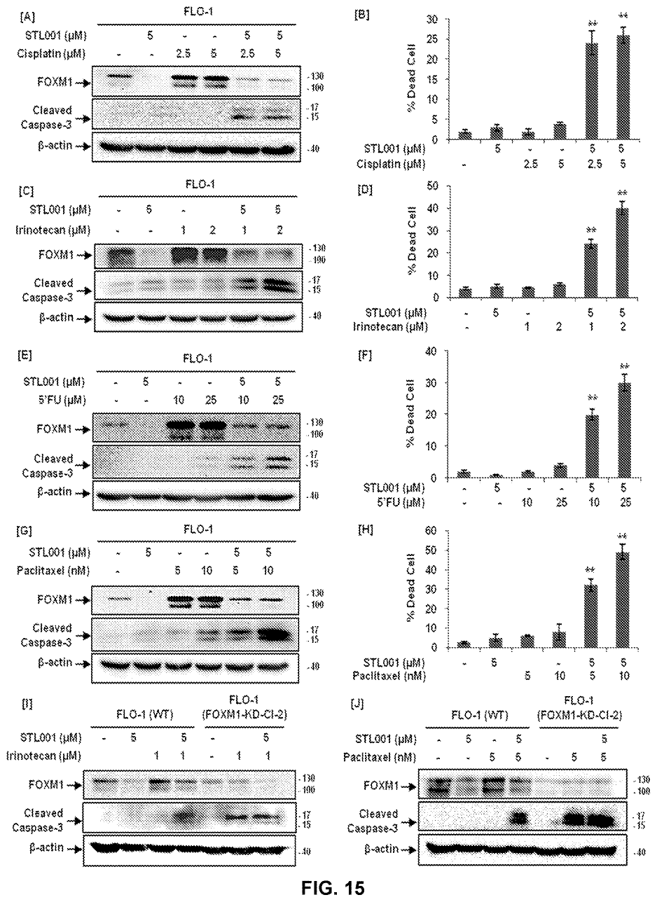

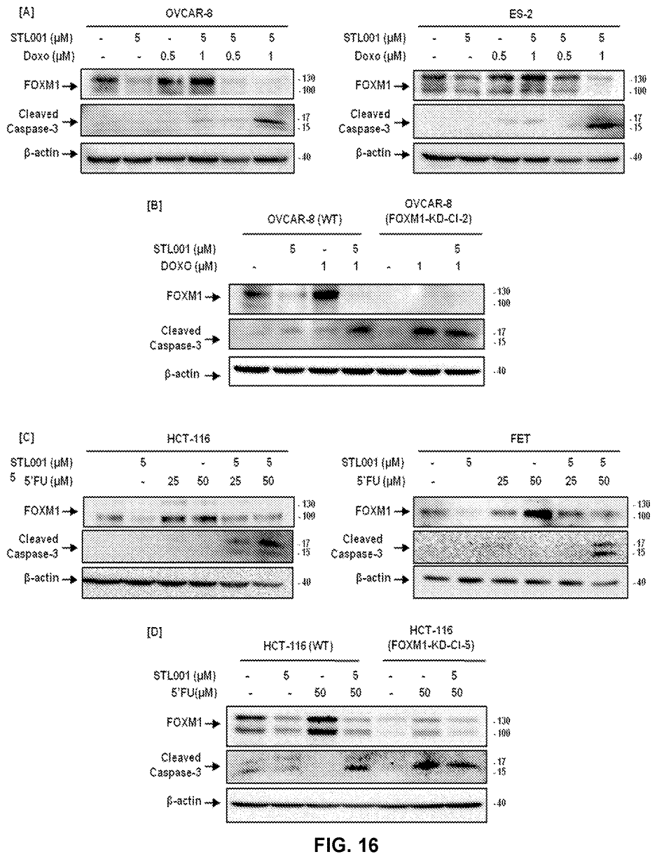

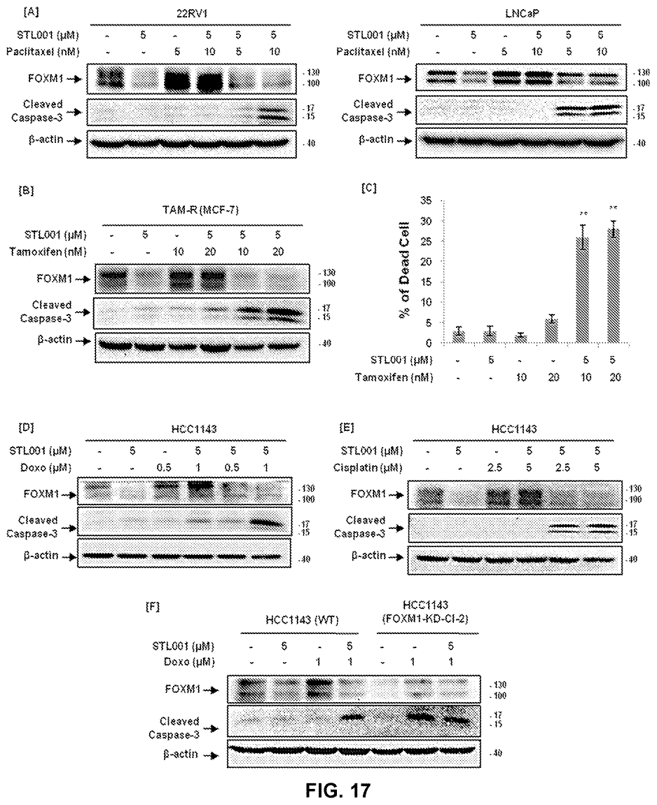

Referring to FIGS. 14-17, STL001 was used in combination with a broad spectrum of drugs of different mechanisms of action: direct DNA-damage (Cisplatin and Doxorubicin), DNA synthesis inhibition (5-FU), mitosis disruption (paclitaxel), or a selective estrogen-receptor (ER) modulator (Tamoxifen). Moreover, the synergy of these drugs was tested with STL001 in model cell lines belonging to solid tumors (such as ovarian cancer, colorectal cancer, breast cancer, prostate cancer) of different etiology.

Doxorubicin is one of the most commonly used anticancer drugs approved by FDA for ovarian cancer, and it is one of the most important drugs used as a second line of chemotherapy for platinum-resistant patients. Ovarian cancer (OVCAR-8, ES-2) cells treated with sub-lethal concentrations of doxorubicin, display a significant increase in FOXM1 protein abundance, whereas addition of STL001 in combination with doxorubicin efficiently prevented FOXM1 activation, resulting in decreased FOXM1 protein levels in comparison with the corresponding control samples. Notably, as a single agent, STL001 did not exert significant cytotoxic effects, but cells treated with doxorubicin chemotherapy in combination with STL001 led to potent induction of apoptotic cell death (indicated by caspase-3 cleavage) when compared with cells treated with doxorubicin chemotherapy alone, results indicate a strong synergistic apoptotic effect of doxorubicin chemotherapy in combination with STL001. Further, it was assessed whether STL001 sensitizes ovarian cancer cells to doxorubicin chemotherapy via mechanisms besides FOXM1 suppression. To test this, OVCAR-8 cells with stable shRNA-mediated FOXM1-knockdown (FOXM1-KD) were used. As expected, FOXM1-deficient OVCAR-8 cells showed increased sensitivity to doxorubicin, detected by potent induction of apoptosis indicated by caspase-3 cleavage; however, the sensitization effect of STL001 was absent in OVCAR-8 cells with stable FOXM1-KD. These findings suggest that FOXM1 is a central factor in ovarian cancer resistance to doxorubicin chemotherapy and mediates the effects of STL001 in sensitization of ovarian cancer cells.

While doxorubicin is well-known to interact and induce DNA damage directly, 5′FU induces indirect DNA damage in cancer cells via interfering with thymidine nucleotide synthesis process. 5′FU is one of the most frequently used chemotherapy for the treatment of solid cancers. Also it is the main first-line chemotherapy used for colorectal cancers; however, resistance to 5′FU therapy exists, resulting a low 5-year survival rate. Similar to doxorubicin effects, treatment of colorectal cancer cells (HCT-116 and HCT-FET) with 5′FU resulted in FOXM1 upregulation without evident cell death of colorectal cancer cells. Whereas, the combination of STL001 and 5′FU therapy remarkably decreased 5′FU-induced FOXM1 levels and significantly enhanced the sensitivity of colorectal cancer cells to cytotoxic effects of 5′FU therapy. Moreover, stable shRNA-mediated FOXM1-KD in HCT-116 cells showed increased sensitivity to 5′FU therapy, detected by potent induction of apoptosis indicated by caspase-3 cleavage; however, the sensitization effect of STL001 was absent in HCT-116 cells with stable FOXM1-KD. FOXM1 has vital role in 5′FU therapy resistance in colorectal cancer and mediating the synergistic response of STL001 with 5′FU therapy.

Taxanes (paclitaxel or docetaxel) are a different class of chemotherapy drugs that act by binding to tubulins/microtubules and suppressing microtubule dynamics during cell division, paclitaxel and docetaxel are similar in function and widely used to treat a verity of human cancers due to their unique anticancer activity. Clinically, paclitaxel is commonly used as an effective natural antineoplastic drug for the treatment of prostate cancer. However tumor cells develop resistance to paclitaxel, restricting its application for the treatment of cancer patients. In line with our previous results, prostate cancer (22RV1, LNCaP) cells treated with paclitaxel (Taxol) at sublethal concentrations showed a prominent increase in cellular FOXM1 protein levels without showing any cytotoxic effects. However, the treatment of prostate cancer cells with STL001 in combination with taxol enhanced the cytotoxic effects of taxol-therapy, detected by induction of strong apoptotic cell death indicated by caspase-3 cleavage. The synergy between STL001 and the taxol-chemotherapy indicates that the functional role of FOXM1 as an inducer of drug resistance is not limited to DNA damage response (DDS) regulation and can be much-more universal.

Breast cancer is the most commonly diagnosed cancer in women, about 80% of all breast cancers are positive for estrogen-receptors (ER+). Many studies have shown that FOXM1 is highly expressed in different types of breast cancer and its expression was closely associated with poor prognosis and chemotherapy resistance in breast cancer patients. Currently, endocrine therapy is a major treatment option for ER+ breast cancer. Tamoxifen is a selective estrogen receptor modulator (SERM), and it is commonly used to treat all stages of hormone dependent or ER+ breast cancers, however, the efficacy of tamoxifen as a breast cancer therapy is not satisfactory because of the development of resistance to tamoxifen. In this context we determine the role of FOXM1 in tamoxifen resistance, in present work, tamoxifen treatment of TAMR cells resulted in FOXM1 upregulation without prominent cell death induction. Whereas, the combination of tamoxifen and STL001 efficiently prevents tamoxifen induced FOXM1 upregulation and drastically enhances the cytotoxic effects of tamoxifen therapy, detected by induction of strong apoptosis indicated by caspase-3 cleavage and loss of TAMR cell viability. The results have confirmed that the combination of tamoxifen plus STL001 could restore the sensitivity to tamoxifen in tamoxifen therapy resistant ER+ breast cancers.

Besides, it will be of interest to test STL001 in combination treatments to target triple-negative breast cancer (TNBC). TNBC is an aggressive form and accounts for 15-20% of all breast cancers. TNBC don't have estrogen or progesterone receptors and the protein called HER2. However, FOXM1 is highly upregulated in TNBC and have significant role in drug resistance of TNBC. Considering this fact, TNBC (HCC-1143) cells were treated with direct DNA-damaging agents (Cisplatin and Doxorubicin) at sub-lethal concentrations showed significantly higher levels of cellular FOXM1 protein without showing prominent cytotoxic effects. However, combination with STL001 efficiently prevents cisplatin or doxorubicin chemotherapy induced FOXM1 upregulation and drastically enhances the cytotoxic effects of both the DNA-damaging agents, detected by induction of strong apoptotic cell death indicated by caspase-3 cleavage. These results indicate that STL001 has a strong synergy with DNA-damaging agents (cisplatin and doxorubicin) to induce pro-apoptotic effects in TNBC.

STL001 was assessed further to verify if STL001 can sensitize TNBC cells through other mechanisms besides FOXM1 suppression. To study this, we used TNBC (HCC-1143) cells with stable shRNA-mediated FOXM1-KD. As expected, HCC-1143 cells with FOXM1-KD show increased sensitivity to doxorubicin, detected by potent induction of apoptosis indicated by caspase-3 cleavage; however, the sensitization effect of STL001 was absent in FOXM1 deficient HCC-1143 cells, suggesting that FOXM1 is the main mediator of STL001 effects on TNBC chemoresistance. In this study, we found that STL001 was effective in sensitizing a wide variety of cancer cells to a broad spectrum of drugs though FOXM1 downregulation, suggesting that the role of FOXM1 in chemoresistance is much more universal.

RNA-seq analysis of the effects of STL001 and FOXM1-KD on global FOXM1 regulatory network: STL001 is a novel small molecule inhibitor of FOXM1, we have examined that STL001 is very effective in sensitizing cancer cells though FOXM1 downregulation; however, the biological activities and the possibility that STL001 sensitize cancer cells through a non-specific FOXM1-independent mechanism are not evaluated. In this perspective, we used RNA-seq to examine the effects of STL001 on gene regulation more globally. To achieve that, we analyzed the patterns of gene expression in OVCAR-8 cells treated with STL001 and cell with stable shRNA-mediated FOXM1 knockdown via full transcriptome RNA-seq and compared with control, untreated OVCAR-8 cells. Out of 16275 protein-coding genes evaluated, a set of 830 and 2502 genes were identified showing highly significant (2-fold or more) differential expression (DE) in STL001 and FOXM1-KD experimental model, respectively. In STL001 group, 535 genes are upregulated and 295 genes are repressed in OVCAR-8 cells, whereas in FOXM1-KD group, 1497 and 1005 genes are up- and down-regulated, respectively. It was found that 204 up-regulated DE-genes and 79 down-regulated DE-genes were common in the STL001 and FOXM1-D group Gene expression changes in STL001 treatment and in FOXM1-KD shows correlation between DE-genes by STL001 treatment and FOXM1-KD (Spearman's rho=0.42). This overlap of transcriptomic effects caused by either STL001 or FOXM1-KD confirms the idea that the effects of STL001 are through suppression of FOXM1 activity.

Further, to verify STL001 selectivity toward FOXM1 regulatory pathways, Gene-Set Enrichment Analysis (GSEA) was performed for “STL001 signature” genes (830 DE-genes) and 2502 DE-genes in FOXM1-KD using canonical pathway gene signatures database, PID. In GSEA analysis of DE-genes of STL001 or FOXM1-KD group, 3 gene-sets were significantly enriched in both the groups. Two gene-sets (PID_AURORA_B_Pathway and PID_FOXM1_Pathway) displayed negative normalized enrichment scores, predicting inactivation of these pathways by STL001 or FOXM1-KD. However, only one gene-set (PID_NFAT_TFpathway) displayed positive normalized enrichment score.

The FOXM1 pathway in PID is a predefined collection of FOXM1 transcription factor network that involved in cell cycle regulation and DNA damage repair, and it promotes tumor cell proliferation. A total of 40 genes from 7 different gene families are engaged in this pathway, including tumor suppressors, the oncogenes, genes encoding cyclins and cyclin-dependent kinases, different transcription factors, and protein kinases, e.g., such as PLK1 and AURKB, as well as FOXM1 itself. FOXM1 pathway is the top enriched pathway in many human cancers. It is noteworthy that PID_AURORA_B_Pathway which is involved in proliferation of cancer cells by positive regulation of cell cycle and G2/M phase transition also represents the activity of direct FOXM1 downstream effectors. Moreover, some of the stress response genes involved in PID_NFAT_TFpathway can be affected by FOXM1 expression, implying that the pathways affected by STL001 or FOXM1-KD converge to the FOXM1 regulated protein network. Taken together, this data show a very high probability of FOXM1 being the main mediator of STL001 effects on gene expression program in cancer cells.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments.

1. A compound having the structure: 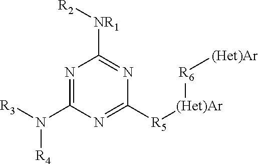
wherein,
(Het)Ar is (hetero)aryl;
R₁ is: H or alkyl, e.g., C₁₋₆ alkyl or C₁₋₃ alkyl;
R₂ is: optionally substituted (hetero)aryl;
R₃ and R₄ are independently: H, Me, Et, cyclopropyl, or R₃ and R₄ taken together are:
—CH₂CH₂—O—CH₂CH₂—, —CH₂CH₂—N(H)—CH₂CH₂—, —CH₂CH₂—S—CH₂CH₂—, —CH₂CH₂—N(Me)-CH₂CH₂—, —CH₂CH₂—N(Et)-CH₂CH₂—, —CH₂CH₂—S(O)—CH₂CH₂—, —CH₂CH₂—S(O)₂—CH₂CH₂—, —CH₂CH₂—N(H)—CH₂CH₂CH₂—, or —CH₂CH₂—N(Me)-CH₂CH₂CH₂—; and
R₅ and R₆ are, independently, saturated or unsaturated (hetero)cyclopentyl having the structure 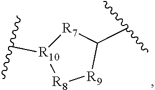
where R₇, R₈, R₉, and R₁₀ are, independently, C, O, N, or S, optionally comprising one or two double bonds in the (hetero)cyclopentyl ring;
or
an amide bond, e.g., 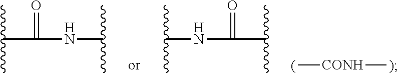
an ester bond, e.g. 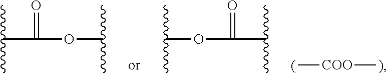
or a pharmaceutically-acceptable salt thereof, including stereoisomers thereof and mixtures of stereoisomers thereof.

2. The compound of claim 1, wherein R₂ is substituted phenyl, thiophenyl, oxazolyl, thiazolyl, isothiazolyl, furanoyl.

3. The compound of claim 1, wherein R₂ is substituted with one or more of: —H, -D, -Me, —CHD₂, —CD₃, CH₂D, —F, —CF₃, —Cl, —CN, -Et, -iPr, or —OMe, or a combination of any of the preceding.

4. The compound of any one of claims 1-3, wherein R₁ is H.

5. The compound of claim 1, having the structure: 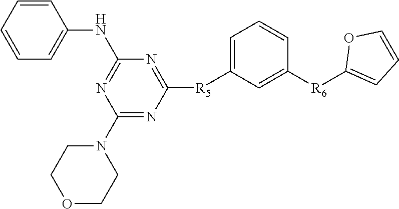
wherein,
R₅ is:
saturated or unsaturated cyclopentyl or heterocyclopentyl having the structure 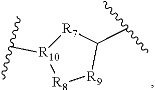
where R₇, R₈, R₉, and R₁₀ are, independently, C, O, N, or S; and
R₆ is:
saturated or unsaturated cyclopentyl or heterocyclopentyl having the structure 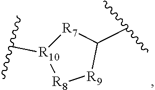
where R₇, R₈, R₉, and R₁₀ are, independently, C, O, N, or S;
an amide bond; or
an ester bond,
or a pharmaceutically-acceptable salt thereof, including stereoisomers thereof and mixtures of stereoisomers thereof.

6. The compound of claim 5, wherein R₅ is

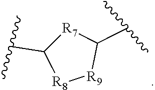

7. The compound of claim 6, wherein at least one of R₇, R₈, and R₉ are N, S, or O.

8. The compound of claim 5 or 6, wherein at least one of R₇, R₈, and R₉ are N.

9. The compound of any one of claims 1-5, wherein for R₅, at least one of R₇, R₈, R₉, and R₁₀ are N, S, or O.

10. The compound of any one of claims 1-5, wherein R₆ is 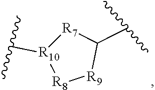
and at least one of R₇, R₈, R₉, and R₁₀ are N, S, or O.

11. The compound of any one of claims 1-5, wherein R₆ is

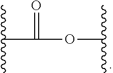 [SMILES: *OC(*)=O]

12. The compound of any one of claims 1-5, wherein R₆ is

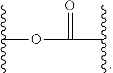 [SMILES: *OC(*)=O]

13. The compound of any one of claims 1-5, wherein R₆ is

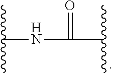 [SMILES: *NC(*)=O]

14. The compound of any one of claims 1-5, wherein R₆ is

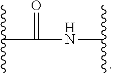 [SMILES: *NC(*)=O]

15. The compound of any one of claims 1-14, wherein R₅ is

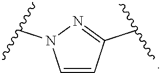 [SMILES: *c1ccn(*)n1]

16. The compound of any one of claim 1-14, wherein R₅ is a pyrazole ring.

17. The compound of any one of claims 1-14, wherein R₅ is a pyrrole, furan, thiophene, pyrazole, imidazole, oxazole, isooxazole, pyrrolidine, thiolane (tetrahydrothiophene), or tetrahydrofuran ring.

18. The compound of claim 1, having the structure: 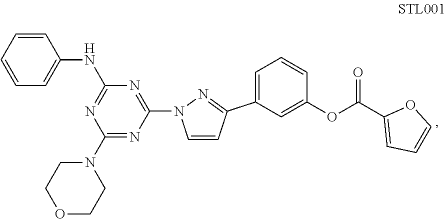 [SMILES: O=C(Oc1cccc(-c2ccn(-c3nc(Nc4ccccc4)nc(N4CCOCC4)n3)n2)c1)c1ccco1]
or a pharmaceutically-acceptable salt thereof, including stereoisomers thereof and mixtures of stereoisomers thereof.

19. A composition comprising the compound of any one of claims 1-18, and a pharmaceutically-acceptable excipient or carrier.

20. The composition of claim 19, in the form of a parenteral dosage form.

21. The composition of claim 19 or 20, further comprising a chemotherapeutic agent.

22. The composition of claim 21, wherein the chemotherapeutic agent is one or more of: abiraterone acetate, altretamine, amsacrine, anhydro vinblastine, auristatin, bafetinib, bexarotene, bicalutamide, BMS 184476, 2,3,4,5,6-pentafluoro-N-(3-fluoro-4-methoxyphenyl)benzene sulfonamide, bleomycin, bosutinib, busulfan, cachectin, cemadotin, chlorambucil, cyclophosphamide, 3′,4′-didehydro-4′-deoxy-8′-norvin-caleukoblastine, docetaxol, doxetaxel, carboplatin, carmustine (BCNU), chlorambucil, cisplatin, cryptophycin, cyclophosphamide, cytarabine, dacarbazine (DTIC), dactinomycin, daunorubicin, decitabine dolastatin, doxorubicin (adriamycin), etoposide, etoposide phosphate, 5-fluorouracil, finasteride, flutamide, gemcitabine, hydroxyurea, hydroxyureataxanes, ifosfamide, imatinib, irinotecan, liarozole, lonidamine, lomustine (CCNU), Enzalutamide, mechlorethamine (nitrogen mustard), melphalan, mitoxantrone, mivobulin isethionate, rhizoxin, sertenef, streptozocin, mitomycin, methotrexate, nilotinib, nilutamide, onapristone, oxaliplatin, paclitaxel, ponatinib, prednimustine, procarbazine, stramustine phosphate, tamoxifen, tasonermin, taxol, teniposide, topotecan, tretinoin, venetoclax, vinblastine, vincristine, vindesine sulfate, vinflunine, or a pharmaceutically acceptable salt or ester thereof.

23. A method of treating a patient having a cancer, comprising administering to the patient amounts of a chemotherapeutic agent and a compound of any one of claims 1-18 or STL427944 where the patient has a leukemia, effective to treat the cancer in the patient.

24. The method of claim 23, wherein the chemotherapeutic agent is one or more of: abiraterone acetate, altretamine, amsacrine, anhydro vinblastine, auristatin, bafetinib, bexarotene, bicalutamide, BMS 184476, 2,3,4,5,6-pentafluoro-N-(3-fluoro-4-methoxyphenyl)benzene sulfonamide, bleomycin, bosutinib, busulfan, cachectin, cemadotin, chlorambucil, cyclophosphamide, 3′,4′-didehydro-4′-deoxy-8′-norvin-caleukoblastine, docetaxol, doxetaxel, carboplatin, carmustine (BCNU), chlorambucil, cisplatin, cryptophycin, cyclophosphamide, cytarabine, dacarbazine (DTIC), dactinomycin, daunorubicin, decitabine dolastatin, doxorubicin (adriamycin), etoposide, etoposide phosphate, 5-fluorouracil, finasteride, flutamide, gemcitabine, hydroxyurea, hydroxyureataxanes, ifosfamide, imatinib, irinotecan, liarozole, lonidamine, lomustine (CCNU), Enzalutamide, mechlorethamine (nitrogen mustard), melphalan, mitoxantrone, mivobulin isethionate, rhizoxin, sertenef, streptozocin, mitomycin, methotrexate, nilotinib, nilutamide, onapristone, oxaliplatin, paclitaxel, ponatinib, prednimustine, procarbazine, stramustine phosphate, tamoxifen, tasonermin, taxol, teniposide, topotecan, tretinoin, venetoclax, vinblastine, vincristine, vindesine sulfate, vinflunine, or a pharmaceutically acceptable salt or ester thereof.

25. The method of claim 23 or 24, wherein the compound is STL001, or a pharmaceutically-acceptable salt thereof, including stereoisomers thereof and mixtures of stereoisomers thereof.

26. The method of any one of claims 23-25, wherein the cancer is a leukemia.

27. The method of claim 26, wherein the leukemia is AML.

28. The method of any one of claims 23-25, wherein the cancer is ovarian cancer, breast cancer, prostate cancer, hepatoma, angiosarcoma, colorectal cancer, melanoma, lung cancer, or gastric cancer and/or FOXM1 is overexpressed or abnormally expressed in cancer cells of the patient.

29. The method of any one of claims 23-25, wherein the cancer is:
ovarian cancer and the chemotherapeutic agent is doxorubicin;
colon cancer, and the chemotherapeutic agent is 5′-fluorouracil;
prostate cancer and the chemotherapeutic agent is paclitaxel;
tamoxifen-resistant breast cancer and the chemotherapeutic agent is tamoxifen; or
triple negative breast cancer and the chemotherapeutic agent is doxorubicin and/or cisplatin, and
wherein FOXM1 is overexpressed or abnormally expressed in cancer cells of the patient.

30. A method of increasing sensitivity of a cancer cell to a chemotherapeutic agent in a patient, e.g., having a leukemia, comprising administering to the patient an amount of a compound as claimed in any one of claims 1-18 or STL427944 where the patient has a leukemia, effective to increase sensitivity of a cancer cell in the patient to the chemotherapeutic agent.

31. The method of claim 30, wherein the cancer cell is a leukemia cell.

32. The method of claim 31, wherein the cancer cell is from a patient having AML (Acute Myeloid Leukemia), such as a cytogenetically normal-AML, such as with FLT3-WT and mutant NPM1.

33. The method of claim 30, wherein the cancer cell is a cancer cell of a patient having ovarian cancer, breast cancer, prostate cancer, hepatoma, angiosarcoma, colorectal cancer, melanoma, esophageal cancer, lung cancer, or gastric cancer and/or FOXM1 is overexpressed or abnormally expressed in cancer cells of the patient.

34. The method of claim 33, wherein the cancer is:
ovarian cancer and the chemotherapeutic agent is doxorubicin;
colon cancer, and the chemotherapeutic agent is 5′-fluorouracil;
prostate cancer and the chemotherapeutic agent is paclitaxel;
tamoxifen-resistant breast cancer and the chemotherapeutic agent is tamoxifen; or
triple negative breast cancer and the chemotherapeutic agent is doxorubicin and/or cisplatin, and
wherein FOXM1 is overexpressed or abnormally expressed in the cancer cells.

35. The method of claim 30, wherein the cancer cell is a solid tumor cancer cell.

36. The method of any one of claims 30-35, wherein the compound is STL001, or a pharmaceutically-acceptable salt thereof, including stereoisomers thereof and mixtures of stereoisomers thereof.

37. The method of any one of claims 30-33, wherein the chemotherapeutic agent is one or more of: abiraterone acetate, altretamine, amsacrine, anhydro vinblastine, auristatin, bafetinib, bexarotene, bicalutamide, BMS 184476, 2,3,4,5,6-pentafluoro-N-(3-fluoro-4-methoxyphenyl)benzene sulfonamide, bleomycin, bosutinib, busulfan, cachectin, cemadotin, chlorambucil, cyclophosphamide, 3′,4′-didehydro-4′-deoxy-8′-norvin-caleukoblastine, docetaxol, doxetaxel, carboplatin, carmustine (BCNU), chlorambucil, cisplatin, cryptophycin, cyclophosphamide, cytarabine, dacarbazine (DTIC), dactinomycin, daunorubicin, decitabine dolastatin, doxorubicin (adriamycin), etoposide, etoposide phosphate, 5-fluorouracil, finasteride, flutamide, gemcitabine, hydroxyurea, hydroxyureataxanes, ifosfamide, imatinib, irinotecan, liarozole, lonidamine, lomustine (CCNU), Enzalutamide, mechlorethamine (nitrogen mustard), melphalan, mitoxantrone, mivobulin isethionate, rhizoxin, sertenef, streptozocin, mitomycin, methotrexate, nilotinib, nilutamide, onapristone, oxaliplatin, paclitaxel, ponatinib, prednimustine, procarbazine, stramustine phosphate, tamoxifen, tasonermin, taxol, teniposide, topotecan, tretinoin, venetoclax, vinblastine, vincristine, vindesine sulfate, vinflunine, or pharmaceutically acceptable salts or esters thereof.

38. The method of any one of claims 23-37, wherein the patient is human.

39. A method of treating a patient having a cancer, comprising administering to the patient amounts of a compound of any one of claims 1-18, or STL427944 where the patient has a leukemia, effective to treat the disease in the patient.

40. The method of claim 39, wherein the compound is STL001, or a pharmaceutically-acceptable salt thereof, including stereoisomers thereof and mixtures of stereoisomers thereof.

41. The method of claim 39 or 40, wherein the disease is a cancer.

42. The method of claim 41, wherein the cancer is a leukemia.

43. The method of claim 42, wherein the leukemia is AML.

44. The method of claim 41, wherein the cancer is ovarian cancer, breast cancer, prostate cancer, hepatoma, angiosarcoma, colorectal cancer, melanoma, lung cancer, or gastric cancer and/or FOXM1 is overexpressed or abnormally expressed in cancer cells of the patient.
